(19) United States
(12) Patent Application Publication
(10) Pub. No.: US 2022/0220450 A1
(43) Pub. Date: Jul. 14, 2022

(54) FABRICATION OF A BIOMIMETIC PLATFORM SYSTEM AND METHODS OF USE

(71) Applicant: Cornell University Center for Technology Licensing, Ithaca, NY (US)

(72) Inventors: Jason A. Spector, New York, NY (US); Kristy A. Brown, New York, NY (US)

(21) Appl. No.: 16/648,186

(22) PCT Filed: Sep. 18, 2018
(86) PCT No.: PCT/US2018/051571

§ 371 Date: Mar. 17, 2020

Related U.S. Application Data
(60) Provisional application No. 62/560,331, filed on Sep. 19, 2017.

(51) Int. Cl. C12N 5/071; C12M 3/00; C12M 1/12

(52) U.S. Cl. CPC C12N 5/0697; C12M 21/08; C12M 25/14; C12N 2533/54; C12N 2502/1305; C12N 2502/30; C12N 2503/04; C12N 2502/095

(57) ABSTRACT

The present technology relates to three-dimensional biomimetic platforms for culturing patient specific cells and tissues in biological material that closely recapitulates the native in vivo environment. The platforms of the present technology enable rapid and flexible biochemical, genomic, and metabolic analysis using a wide variety of assays, and live or end-point biological imaging.

### CROSS-REFERENCES TO RELATED APPLICATIONS

This application claims the benefit of and priority to U.S. Provisional Application 62/560,331 filed Sep. 19, 2017, which is hereby incorporated by reference in its entirety.

### TECHNICAL FIELD

The present technology relates to the field of tissue engineering, including three-dimensional biomimetic platform systems that recapitulate the native in vivo environment and are useful for culturing patient specific cells and tissues.

### BACKGROUND

The following description of the background of the present technology is provided simply as an aid in understanding the present technology and is not admitted to describe or constitute prior art to the present technology.

Artificial human tissues have the potential to revolutionize the medical field by facilitating rapid drug screening as well as basic biology research. For instance, diagnosis and treatment of disease could be rapidly accomplished in a small-scale clinical environment before moving on to human subjects, thus potentially saving lives and financial resources.

Human tissues have a complex structure with multiple, interacting cell types, which in turn depend on adjacent vascular and lymphatic vessel networks for their survival and function. See e.g., FIG. 7 (showing the complex spatial arrangement of endothelial cells (HUVEC), smooth muscle cells (HASMC), and pericytes (HPLP) within a vascular structure). For instance, blood vessels generally consist of three layers, each with its own unique structure and composition. The thinnest layer alone consists of a single layer of simple squamous endothelial cells glued by a polysaccharide intercellular matrix, surrounded by a thin layer of endothelial connective tissue. Other layers include connective tissue, polysaccharide substances, vascular smooth muscle, and nerves. Capillaries, which are the simplest blood vessels, consist of a layer of endothelium and occasional connective tissue.

Similarly, tumors are also very complex structures—while tumors are generally depicted as a solid mass of cells, at the very least they also contain endothelial cells in their own vasculature. This hierarchal complexity has prevented faithful recapitulation of human tissue in an artificial environment.

Fundamental biological questions remain unanswered due to the limitation of existing artificial tissues. For example, the processes of cell fate regulation and differentiation need to be delineated in greater detail. Angiogenesis, the development of new blood vessels, has yet to be precisely elucidated, both during normal development and in disease progression such as tumor growth. Another question relates to cancer metastasis and more specifically how metastasis progresses within a complex vessel network and microenvironment, and what guiding molecules direct tumor cells to target regions.

### SUMMARY

In one aspect, the present disclosure provides a three-dimensional biomimetic platform comprising (a) a biocompatible substrate including collagen, a stromal vascular fraction, adipocytes, and organoids, and (b) patient-specific cells, wherein the patient-specific cells are homogenously or heterogeneously dispersed within the biocompatible substrate. In some embodiments, the biocompatible substrate further comprises lymphatic endothelial cells.

In another aspect, the present disclosure provides a three-dimensional biomimetic platform system comprising (a) a biocompatible substrate including collagen, wherein the biocompatible substrate comprises one or more conduits, and (b) patient-specific cells cultured in the one or more conduits.

Also disclosed herein is a three-dimensional biomimetic platform system comprising (a) a biocompatible substrate including collagen, a stromal vascular fraction, adipocytes, and organoids, wherein the biocompatible substrate comprises one or more conduits, and (b) patient-specific cells cultured in the one or more conduits. In some embodiments, the biocompatible substrate further comprises lymphatic endothelial cells. The stromal vascular fraction may include one or more of adipose-derived stem/stromal cells (ADSCs), endothelial precursor cells (EPCs), endothelial cells (ECs), macrophages, smooth muscle cells, lymphocytes, pericytes, and pre-adipocytes. The organoids may be breast organoids, cerebral organoids, intestinal organoids, gastric organoids, hepatic organoids, lingual organoids, thyroid organoids, thymic organoids, testicular organoids, pancreatic organoid, epithelial organoids, lung organoids, kidney organoids, gastruloids (embryonic organoids), or cardiac organoids.

Additionally or alternatively, in some embodiments, the patient-specific cells are isolated from a subject suffering from a disease or condition (e.g., cancer) or a healthy subject. Examples of patient-specific cells include, but are not limited to, cancerous cells, pre-cancerous cells, pericytes, stem cells, blood cells, immune cells, platelets, central nervous system neurons, glial cells, peripheral nervous system neurons, skeletal muscle cells, smooth muscle cells, chondrocytes, bone cells, skin cells, hepatic cells, endothelial cells, epithelial cells, cardiac cells, pancreatic cells, adipocytes, gastric cells, intestinal cells, renal cells, fibroblasts, gall bladder cells, duct cells, pneumocytes, lens cells, sensory transducer cells, autonomic neurons, gland cells, hormone secreting cells, nurse cells, germ cells, or any combination thereof.

Additionally or alternatively, in some embodiments of the three-dimensional biomimetic platform systems of the present technology, the biocompatible substrate comprises about 0.1 wt % to about 10 wt % of collagen. Additionally or alternatively, in some embodiments of the three-dimensional biomimetic platform systems of the present technology, the collagen of the biocompatible substrate is a Type I collagen, a Type II collagen, a Type III collagen, a Type IV collagen, a Type V collagen, a Type VI collagen, a Type VII collagen, a Type VIII collagen, a Type IX collagen, a Type X collagen, a Type XI collagen, a Type XII collagen, a Type XIII collagen, a Type XIV collagen, a Type XV collagen, a Type XVI collagen, a Type XVII collagen, a Type XVIII collagen, a Type XIX collagen, a Type XX collagen, a Type XXI collagen, a Type XXII collagen, a Type XXIII collagen, a Type XXIV collagen, a Type XXV collagen, a Type XXVI collagen, a Type XXVII collagen, a Type XXVIII collagen, a Type XXIX collagen, or any mixture thereof. In any of the embodiments disclosed herein, the collagen may be modified with a glycosylating agent. Examples of glycosylating agents include, but are not limited to glucose, ribose, fructose, galactose, glucose-6-Phosphate, lactose, maltose, xylose, glyceraldehyde, glutaraldehyde, cellobiose, corn syrup, maltodextrin, dextrin, as well as any other glycosylating agents known in the art. In any of the embodiments disclosed herein, the collagen has an elastic compressive modulus that ranges from about 3 kPa to about 40 kPa.

Additionally or alternatively, in some embodiments of the three-dimensional biomimetic platform systems of the present technology, the biocompatible substrate further comprises at least one non-collagen extracellular matrix component. Examples of non-collagen extracellular matrix components include, but are not limited to, fibronectin, laminin, hyaluronic acid, Matrix-bound nanovesicles (MBVs), elastin, proteoglycans, glycosaminoglycans (GAGs), heparan sulfate, perlecan, agrin, chondroitin sulfate, and keratan sulfate.

In any of the embodiments of the three-dimensional biomimetic platform systems disclosed herein, the one or more conduits have a shape selected from the group consisting of straight, curved, U-shape, zigzagged or any combination thereof. In certain embodiments, the one or more conduits form a vascular channel. Additionally or alternatively, in some embodiments, the one or more conduits may arborize and/or coalesce into a vascular network.

In another aspect, the present disclosure provides a method for producing a biomimetic platform system of the present technology comprising (a) preparing a biocompatible substrate, (b) embedding a sacrificial material within the biocompatible substrate, (c) degrading the sacrificial material to produce one or more conduits within the biocompatible substrate, and (d) applying patient-specific cells to the one or more conduits within the biocompatible substrate. In some embodiments, the method further comprises culturing the patient-specific cells under conditions that permit maturation of the patient-specific cells in the biocompatible substrate. The biocompatible substrate may be any polymer suitable for culturing cells, providing a medium for the cells to attach to or providing a suitable environment for a cell suspension. In any of the above embodiments of the methods disclosed herein, the biocompatible substrate comprises at least one collagen (e.g., Type I collagen, Type II collagen, Type III collagen, Type IV collagen, Type V collagen). Additionally or alternatively, in certain embodiments, the biocompatible substrate further comprises one or more components selected from the group consisting of stromal vascular fraction, adipocytes, and organoids.

Examples of suitable sacrificial materials include, but are not limited to, poloxamers, shellac, carbohydrate glass, polyvinyl alcohol (PVA), and gelatin microparticles. Examples of poloxamers include, but are not limited to poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 Benzoate, and poloxamer 182 dibenzoate. In some embodiments, the sacrificial material is poloxamer 407 (e.g., Pluronic® F127).

Additionally or alternatively, in some embodiments, the method further comprises adding one or more biomolecules to the biocompatible substrate to promote cell culture and cell viability (e.g., growth factors, blood, plasma, hormones, cytokines, enzymes, vitamins, fatty acids, lymphokines, and the like).

In one aspect, the present disclosure provides a method for monitoring at least one biological activity of patient-specific cells ex vivo comprising (a) culturing patient-specific cells in a biomimetic platform system of the present technology under conditions that permit maturation of the patient-specific cells; and (b) assaying at least one biological activity of the patient-specific cells. Additionally or alternatively, in some embodiments, the method further comprises implanting mature patient-specific cells from the biomimetic platform system into a host organism (e.g., a rodent such as a mouse or a rat). In certain embodiments, the implanted mature patient-specific cells are anastomosed to and perfused by the circulatory system of the host organism. Examples of suitable biological activities include, but are not limited to cell viability, cell growth, cell division, apoptosis, cell migration, angiogenesis, gene expression, blood coagulation, metastasis etc. The patient-specific cells may comprise any one or more cell types disclosed herein.

In one aspect, the present disclosure provides a method for screening the effect of a candidate agent on patient-specific cells comprising (a) contacting the candidate agent with a biomimetic platform system of the present technology, wherein the biomimetic platform system comprises patient-specific cells that are cultured under conditions that permit maturation of the patient-specific cells, and (b) assaying at least one biological activity of the treated patient-specific cells. In some embodiments, the treated patient-specific cells exhibit an alteration in at least one biological activity compared to that observed in untreated patient-specific cells. Examples of suitable biological activities include, but are not limited to cell viability, cell growth, cell division, apoptosis, cell migration, angiogenesis, gene expression, blood coagulation, metastasis etc. The patient-specific cells may comprise any one or more cell types disclosed herein.

In another aspect, the present disclosure provides a method for evaluating the toxicity of a candidate agent on patient-specific cells obtained from a healthy subject comprising (a) contacting the candidate agent with a biomimetic platform system of the present technology, wherein the biomimetic platform system comprises patient-specific cells that are cultured under conditions that permit maturation of the patient-specific cells, (b) assaying the viability of the treated patient-specific cells, and (c) determining that the candidate agent is toxic when the treated patient-specific cells exhibit decreased viability compared to that observed in untreated patient-specific cells.

In one aspect, the present disclosure provides a method for determining the therapeutic efficacy of a candidate agent for treating a disease (e.g., cancer) in a patient in need thereof comprising (a) contacting a biomimetic platform system of the present technology with the candidate agent, wherein the biomimetic platform system comprises patient-specific diseased cells that are cultured under conditions that permit maturation of the patient-specific diseased cells, and (b) determining that the candidate agent is therapeutically effective when the treated patient-specific diseased cells exhibit decreased viability compared to that observed in untreated patient-specific cells. In certain embodiments, the disease is a cancer selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

In some embodiments of the methods disclosed herein, the candidate agent may be a synthetic low-molecular-weight compound, a natural compound, a recombinant protein, a purified or crude protein, a peptide, a non-peptide compound, an antibody, an engineered cell, a vaccine, a nucleic acid (e.g., a siRNA, an antisense oligonucleotide, a sgRNA, an aptamer), a recombinant virus, a recombinant microorganism, a ribozyme, a cell extract, a cell culture supernatant, a microbial fermentation product, a marine organism extract, a plant extract, or any combination thereof. In some embodiments, the candidate agent is a chemotherapeutic agent.

### BRIEF DESCRIPTION OF THE DRAWINGS

FIG. 1 shows a schematic for fabricating an illustrative embodiment of the biomimetic platform system of the present technology using Type I collagen and sacrificial material (e.g., Pluronic® F127).

FIG. 2 shows a schematic for seeding an illustrative embodiment of the biomimetic platform system of the present technology with patient-specific cells.

FIG. 3 is a schematic representation of different types of flow that happen based on shape and size of vessels.

FIG. 4 shows a Pluronic® F127 microfiber (with regions of different shear stresses) that was generated using the AnsysFluent fluid simulation software (Canonsburg, Pa.). Acute angles were predicted to provide regions of high shear stress and linear regions were predicted to provide regions of lower shear stress.

FIG. 5 shows the generation of a conduit with the desired shape within the biomimetic platform using the Pluronic® F127 sacrificial methods described in Example 1. The conduit was seeded with smooth muscle cells and endothelial cells.

FIG. 6A shows hematoxylin and eosin (H&E) staining of a vascular structure with lumen after 14 days of culture.

FIG. 6B shows a fluorescent microscopic image of CD31 positive endothelial cells (arrows) lining the walls of the channel after 14 days of culture.

FIG. 7 shows a confocal microscopy image of a vascular structure with endothelial cells (HUVEC; white arrow), smooth muscle cells (HASMC; arrowhead), and pericytes (HPLP; double-stem arrow), at 50 μm (top panel) and 25 μm (bottom panel).

FIG. 8 shows the distribution of pericytes relative to the wall of the vessel/vascular structure (3D fluorescent microscopy model (bottom); black and white image rendition (top) of the same).

FIG. 9A is a graph that plots the number of pericytes based on their distance from the neovessel.

FIG. 9B shows the distribution of pericytes in a vessel with shear stress compared to the distribution of pericytes in a control vessel with no induced shear stress.

FIG. 9C shows a distribution of pericyte distance from the vessel wall as a function of time.

FIG. 10A shows H&E staining of the biomimetic platform system including 15% v/v adipocytes and organoids after 3 days.

FIG. 10B shows a microscopic image of normal epithelial breast cells cultured with the 3D-breast component biomimetic platform system.

FIG. 11A shows a confocal microscopy image of an organoid.

FIG. 11B shows a confocal microscopy image of the 3D-breast component biomimetic platform system.

FIG. 11C is an overlay of a confocal microscopy image with a brightfield microscopy image showing an organoid, cancer cells, and adipocytes within the 3D-breast component biomimetic platform system.

FIG. 11D shows a rendition of the z-stacks of the 3D-breast component biomimetic platform system of the present technology.

FIG. 12 shows a fluorescence microscopy image demonstrating lipid transfer on 0.6% collagen with 15% v/v adipocytes, 2 million/mL SVF and MDA-MBA231 cancer cells. (Hoechst—white arrow; BODIPY—arrowhead; Cytokeratin 19—double stem arrow).

FIG. 13A shows a diagram of the biomimetic platform system including cancer hemispheroid, organoids, adipocytes, and SVF (unlabeled cells in bulk).

FIG. 13B shows H&E staining of the biomimetic platform system with adipocytes, stromal cells, and breast duct organoid.

FIG. 13C shows a confocal microscopy image of the biomimetic platform system with fluorescently tagged cancer cell button, adipocytes, and organoids+SVF.

FIG. 14 shows a confocal microscopy image of the biomimetic platform system with 25% v/v adipocytes, SVF, and organoids (DAPI—white arrow; BODIPY—arrowhead; Cytokeratin 19—double stem arrow).

FIG. 15 shows the mechanical properties of collagen hydrogels of the biomimetic platform. The elastic compressive moduli of the collagen gels were measured at different protein densities (w/v). Data are presented as mean±standard deviation.

FIG. 16A shows confocal reflectance microscopy images of collagen hydrogels that were dosed with a 0, 100, or 200 mM ribose solution. Scale bar—15 μm.

FIG. 16B depicts the average pore area (%) of collagen hydrogels that were dosed with a 0, 100, or 200 mM ribose solution.

FIG. 16C depicts the average pore diameter (μall) of collagen hydrogels that were dosed with a 0, 100, or 200 mM ribose solution.

FIG. 16D shows the mechanical properties of collagen hydrogels after ribose dosing. The elastic compressive moduli of 3 mg/mL collagen hydrogels were measured after being dosed with a 0, 100, or 200 mM ribose solution. Data are presented as mean±standard deviation.

FIG. 17A shows multiphoton microscopic images of (i) non-cancer containing constructs and (ii)-(iii) cancer containing constructs with fluorescently tagged HUVEC, and HASMC cells. The cancer containing constructs exhibited invasion of the MDA-MB231 cells (white arrow) toward the lumen of the neovessel which disrupts the endoluminal lining (arrowhead) and the sub-adjacent smooth muscle cells (double-stem arrow). Scale bar—100 μm.

FIG. 17B is a schematic showing the placement of a cancer cell spheroid in an embodiment of the collagen only biomimetic platform system disclosed herein, and the progression of cancer metastasis in the mechanically tuned microenvironment created by the biomimetic platform system.

FIG. 17C shows an example of a confocal micrograph of tumor-induced angiogenesis in a mechanically tuned (higher elastic compressive modulus) microenvironment (200 μM ribose with MDA-MB231 spheroid). After 10 days, neovessels (10-80 μm in diameter) formed towards the spheroid. MDA-MB231 cancer cells subsequently broke off and invaded the neovessels (circles identify locations of metastasis).

FIG. 18A shows fluorescence images of MDA-MB231 cells after being embedded in collagen polymerized gels of varying matrix stiffness. Scale bar: 150 μm.

FIG. 18B shows the increase in the projected area of MDA-MB231 spheroids over time.

FIG. 18C shows the elastic compressive modulus of collagen gels dosed with 0 mM, 100 mM, and 200 mM ribose solution after incubation with MDA-MB231 spheroids over time.

FIG. 19A shows fluorescence images of collagen gels (0.6% (w/v)) including 15% v/v adipocytes, stromal cells, breast organoids, and 200,000 MDA-MB231 cancer cells labelled with mCherry that were incubated with various concentrations of doxorubicin (0 μM, 0.001 μM, 0.01 μM, 0.1 μM, 10 μM from left to right). Doxorubicin uptake was increased in biomimetic platform systems incubated with high concentrations of doxorubicin (1-10 μM). The large globules of doxorubicin signal observed in platforms on far right of FIG. 19A (white arrows) correspond with doxorubicin uptake by adipocytes.

FIG. 19B shows fluorescence images of 0.6% (w/v) collagen and 200,000 MDA-MB231 cancer cells labelled with mCherry that were incubated with various concentrations of doxorubicin (0 μM, 0.001 μM, 0.01 μM, 0.1 μM, 10 μM from left to right). A decrease in the absolute number of cells (indicated by reduced mCherry and DAPI signals) was observed when the platforms were incubated with 1-10 μM doxorubicin, thus demonstrating the concentration-dependent cytotoxic effects of doxorubicin.

FIG. 19C shows fluorescence images of collagen gels (0.6% (w/v)) including 15% v/v adipocytes, stromal cells, breast organoids, but without mCherry labelled MDA-MB231 cancer cells, that were incubated with various concentrations of doxorubicin (0 μM, 0.001 μM, 0.01 μM, 0.1 μM, 10 μM from left to right). Doxorubicin uptake was increased in biomimetic platform systems incubated with high concentrations of doxorubicin. FIG. 19C demonstrates the permeability of the biomimetic platform and adipocytes to doxorubicin (as evidenced by increased signal) with increasing doxorubicin concentrations (white arrow).

FIG. 20A shows the decreased sensitivity of MDA-MB231 cancer cells to increasing concentrations of doxorubicin (0-10 μM) when cultured in the 3D-breast component biomimetic platform system compared to the 3D-collagen only biomimetic platform system.

FIG. 20B shows the decreased sensitivity of MDA-MB231 cancer cells to increasing concentrations of doxorubicin (0-10 μM) when cultured in the 3D-breast component biomimetic platform system compared to the 3D-collagen only biomimetic platform system.

FIG. 20C shows the decreased sensitivity of MDA-MB468 cancer cells to increasing concentrations of doxorubicin (0-10 μM) when cultured in the 3D-breast component biomimetic platform system compared to the 3D-collagen only biomimetic platform system.

FIG. 21A shows the decreased sensitivity of HS578T cancer cells to increasing concentrations of doxorubicin (0-10 μM) when cultured in the 3D-breast component biomimetic platform system compared to the 3D-collagen only biomimetic platform system.

FIG. 21B shows the decreased sensitivity of HS578T cancer cells to increasing concentrations of doxorubicin (0-10 μM) when cultured in the 3D-breast component biomimetic platform system compared to the 3D-collagen only biomimetic platform system.

FIG. 21C shows the decreased sensitivity of HS578T cancer cells to increasing concentrations of doxorubicin (0-10 μM) when cultured in the 3D-breast component biomimetic platform system compared to the 3D-collagen only biomimetic platform system.

FIG. 21D shows the decreased sensitivity of HS578T cancer cells to increasing concentrations of doxorubicin (0-10 μM) when cultured in the 3D-breast component biomimetic platform system compared to the 3D-collagen only biomimetic platform system.

### DETAILED DESCRIPTION

Current biomimetic platform systems are usually 2D environments that are based on either invasive cell administration into immunodeficient mice or on indirect data from ex vivo tissue/cell analysis. Many ex vivo platforms are aimed at studying very specific processes that take place in living organisms, and fail to incorporate the amount and high degree of complexity of the interactions that occur between the multiple cellular components and the extracellular matrix. Other models have attempted to use advances in engineering to recreate complex interactions by incorporating synthetic materials, or complicated architectures that are not anatomically or physiologically accurate, thereby representing a difficult barrier to overcome when translating into clinical applications. For example, some models use microfluidic membranes in an attempt to replicate membrane diffusion of gases and cellular functions that take place in different organs (Chen, Trends in Cell Biology, 26(11): 798-800 (2016). Some models, like the microwell arrays with methacrylated gelatin and mammary gland components like SVF obtained from mice, have shown promise but lack an extracellular matrix with proteins encountered in vivo and offer no significant advantage over animal models (i.e., extensive studies would still be required to ensure proper translation to human clinical applications).

The biomimetic platform systems disclosed herein overcome the aforementioned obstacles and serves as a 3D model that is physiologically and anatomically accurate. The translational capabilities of the biomimetic platform systems of the present technology are based at least in part on the fact that the primary cells that are used to generate the platform are obtained from a patient. By using the patient's own stromal cells, interactions that may not be mimicked in other models can be observed. The biomimetic platform system disclosed herein permit visualization of interactions between cancer cells with healthy tissue, and utilizes cancer associated stroma which has been increasingly recognized as playing an important role in cancer behavior. The biomimetic platform systems of the present technology incorporate patient adipocytes, and also overcomes the difficulties associated with adipocyte-culture that have been previously reported in other models (see Carswell K A et al., Methods Mol Biol 806:203-214 (2012)).

The biomimetic platform systems of the present technology successfully replicate tissue anatomy ex vivo by including patient derived organoids which permit the reproduction of native cell-to-cell interactions that are observed in vivo. Organoid isolation and culture methods are known in the art, and are described in Aberle et al., BJS 105: e48-e60 (2018), AMSBIO, Organoid Culture Handbook (August 2017), Drost et al., Nat Protoc. 11(2): 347-358 (2016), Weygan et al., J Cancer Prev Curr Res, 8(7): 00307 (2017); Sato T et al., Gastroenterology. 141:1762 (2011), and Meijer et al., Future Sci OA. 3(2): FSO190 (2017), and are herein incorporated by reference in their entirety. Thus, the biomimetic platform systems of the present technology can be adapted for culturing a wide variety of tissues by including tissue-specific organoids, thereby recapitulating the in vivo environment of the distinct tissue types.

In addition to the patient-derived cellular components, the biomimetic platform systems of the present technology comprise Type I collagen, which is predominantly found in the extracellular matrix (ECM) of multiple human tissues, as opposed to utilizing other hydrogels like matrigel or modified gelatin. The biomimetic platform systems of the present technology also comprise sacrificial layers that can be further developed into a vascularized model with and without flow, thus bypassing the need for animal models and is useful for mimicking human tissue behavior and predicting response to various cancer treatments.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “biomimetic platform” refers to a biocompatible substrate comprising an extracellular matrix protein (e.g., Type I collagen). The biomimetic platform may optionally include one or more of the following: extracted organoids, stromal vascular fractions, and adipocytes. The term “biomimetic platform system” refers to a biomimetic platform, wherein the biocompatible substrate comprising the extracellular matrix protein also includes one or more conduits in which patient-specific cells are cultured. In some embodiments, the patient-specific cells are derived from a cancer patient or a healthy patient. As used herein, the terms “conduit,” “channel” and “vessel” are used interchangeably.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “individual”, “patient”, or “subject” are used interchangeably and refer to an individual organism, a vertebrate, a mammal, or a human. In certain embodiments, the individual, patient or subject is a human.

As used herein, the term “organoids” are miniature, self-organized, three-dimensional tissue cultures that are derived from one or few cells from a tissue, embryonic stem cells or induced pluripotent stem cells. Such cultures can be crafted to replicate much of the complexity of an organ, or to express selected aspects of it like producing only certain types of cells. Organoids can self-organize in three-dimensional culture owing to their self-renewal and differentiation capacities.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, a “sample” or “biological sample” refers to a body fluid or a tissue sample isolated from a subject.

As used herein, “stromal vascular fraction” or “SVF” refers to an aqueous cell fraction that is derived from enzymatic digestion of lipoaspirate, and comprises adipose-derived stem/stromal cells (ADSCs), endothelial precursor cells (EPCs), endothelial cells (ECs), macrophages, smooth muscle cells, lymphocytes, pericytes, and pre-adipocytes.

As used herein, “wt %” refers to the percentage of the dry weight of a component over the total volume of the biocompatible substrate present in the biomimetic platform system described herein.

### Biomimetic Platform Systems of the Present Technology

The present disclosure provides three-dimensional biomimetic platform systems that include a biocompatible substrate comprising one or more conduits. The biocompatible substrate may be any substrate suitable for culturing cells, providing a medium for the cells to attach to or providing a suitable environment for a cell suspension. The biomimetic platform systems disclosed herein recapitulate a three-dimensional in vivo tissue and organ environment by creating fully vascularized constructs with vascular and lymphatic vessel networks and epithelialized ducts and channels. See Examples 2-6.

The biomimetic platform systems of the present technology successfully replicate tissue anatomy ex vivo by including patient derived organoids which permit the reproduction of native cell-to-cell interactions that are observed in vivo. Organoid isolation and culture methods are known in the art, and are described in Aberle et al., BJS 105: e48-e60 (2018), AMSBIO, Organoid Culture Handbook (August 2017), Drost et al., Nat Protoc. 11(2): 347-358 (2016), Weygan et al., J Cancer Prev Curr Res, 8(7): 00307 (2017); Sato T et al., Gastroenterology. 141:1762 (2011), and Meijer et al., Future Sci OA. 3(2): FSO190 (2017), and are herein incorporated by reference in their entirety. Thus, the biomimetic platform systems of the present technology can be adapted for culturing a wide variety of tissues by including tissue-specific organoids, thereby recapitulating the in vivo environment of the distinct tissue types.

In one aspect, the present disclosure provides a three-dimensional biomimetic platform comprising (a) a biocompatible substrate including collagen, a stromal vascular fraction, adipocytes, and organoids, and (b) patient-specific cells, wherein the patient-specific cells are homogenously or heterogeneously dispersed within the biocompatible substrate. Additionally or alternatively, in some embodiments, the biocompatible substrate is uniformly solid and lacks any conduits. In some embodiments, the biocompatible substrate further comprises lymphatic endothelial cells.

In another aspect, the present disclosure provides a three-dimensional biomimetic platform system comprising (a) a biocompatible substrate including collagen, wherein the biocompatible substrate comprises one or more conduits, and (b) patient-specific cells cultured in the one or more conduits.

Also disclosed herein is a three-dimensional biomimetic platform system comprising (a) a biocompatible substrate including collagen, a stromal vascular fraction, adipocytes, and organoids, wherein the biocompatible substrate comprises one or more conduits, and (b) patient-specific cells cultured in the one or more conduits. In some embodiments, the biocompatible substrate further comprises lymphatic endothelial cells. The stromal vascular fraction may include one or more of adipose-derived stem/stromal cells (ADSCs), endothelial precursor cells (EPCs), endothelial cells (ECs), macrophages, smooth muscle cells, lymphocytes, pericytes, and pre-adipocytes. The organoids may be breast organoids, cerebral organoids, intestinal organoids, gastric organoids, hepatic organoids, lingual organoids, thyroid organoids, thymic organoids, testicular organoids, pancreatic organoid, epithelial organoids, lung organoids, kidney organoids, gastruloids (embryonic organoids), or cardiac organoids.

Additionally or alternatively, in some embodiments, the patient-specific cells are isolated from a subject suffering from a disease or condition (e.g., cancer) or a healthy subject. Examples of patient-specific cells include, but are not limited to, cancerous cells, pre-cancerous cells, pericytes, stem cells, blood cells, immune cells, platelets, central nervous system neurons, glial cells, peripheral nervous system neurons, skeletal muscle cells, smooth muscle cells, chondrocytes, bone cells, skin cells, hepatic cells, endothelial cells, epithelial cells, cardiac cells, pancreatic cells, adipocytes, gastric cells, intestinal cells, renal cells, fibroblasts, gall bladder cells, duct cells, pneumocytes, lens cells, sensory transducer cells, autonomic neurons, gland cells, hormone secreting cells, nurse cells, germ cells, or any combination thereof. In some embodiments, the patient-specific cells are isolated from a human, a mouse, a rat, a monkey, a cow, a sheep, a horse, or any other animal used in research or agriculture. In yet another embodiment, the patient-specific cells are isolated from a subject in need of a medical procedure or a diagnostic test, and would benefit from a three-dimensional modeling of said subject's organs or tissues.

Additionally or alternatively, in some embodiments, the stromal vascular fraction, adipocytes, and organoids are isolated from a subject suffering from a disease or condition (e.g., cancer) or a healthy subject. In some embodiments, the stromal vascular fraction, adipocytes, organoids, and patient-specific cells are isolated from the same subject. In other embodiments, the stromal vascular fraction, adipocytes, and organoids are isolated from a different subject than the subject from which the patient-specific cells are isolated.

In any of the embodiments of the three-dimensional biomimetic platform systems disclosed herein, the collagen may be selected from the group consisting of a mammalian collagen, a marine collagen, a murine collagen, a porcine collagen, an ovine collagen, an equine collagen, a bovine collagen, a human collagen, an avian collagen, and any combination thereof. The collagen may be isolated from a biological source or recombinantly generated using any suitable method known in the art. Additionally or alternatively, in some embodiments, the collagen may be neutralized with HEPES buffer or NaOH. In any of the embodiments disclosed herein, the collagen may be modified with a glycosylating agent. Examples of glycosylating agents include, but are not limited to glucose, ribose, fructose, galactose, glucose-6-Phosphate, lactose, maltose, xylose, glyceraldehyde, glutaraldehyde, cellobiose, corn syrup, maltodextrin, dextrin, as well as any other glycosylating agents known in the art.

Additionally or alternatively, in some embodiments, the collagen has an average pore diameter of about 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm or any range including and/or in between any two of the preceding values.

Additionally or alternatively, in some embodiments, the collagen has an elastic compressive modulus that ranges from about 3 kPa to about 40 kPa. For example, in any of the embodiments disclosed herein, the collagen has an elastic compressive modulus of about 3 kPa, about 4 kPa, about 5 kPa, about 6 kPa, about 7 kPa, about 8 kPa, about 9 kPa, about 10 kPa, about 11 kPa, about 12 kPa, 13 kPa, about 14 kPa, about 15 kPa, about 16 kPa, about 17 kPa, about 18 kPa, about 19 kPa, about 20 kPa, about 21 kPa, about 22 kPa, 23 kPa, about 24 kPa, about 25 kPa, about 26 kPa, about 27 kPa, about 28 kPa, about 29 kPa, about 30 kPa, about 31 kPa, about 32 kPa, 33 kPa, about 34 kPa, about 35 kPa, about 36 kPa, about 37 kPa, about 38 kPa, about 39 kPa, about 40 kPa or any range including and/or in between any two of the preceding values.

Additionally or alternatively, in some embodiments of the three-dimensional biomimetic platform systems of the present technology, the biocompatible substrate comprises an amount of collagen that is about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3.0 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4.0 wt %, about 4.1 wt %, about 4.2 wt %, about 4.3 wt %, about 4.4 wt %, about 4.5 wt %, about 4.6 wt %, about 4.7 wt %, about 4.8 wt %, about 4.9 wt %, about 5.0 wt %, about 5.1 wt %, about 5.2 wt %, about 5.3 wt %, about 5.4 wt %, about 5.5 wt %, about 5.6 wt %, about 5.7 wt %, about 5.8 wt %, about 5.9 wt %, about 6.0 wt %, about 6.1 wt %, about 6.2 wt %, about 6.3 wt %, about 6.4 wt %, about 6.5 wt %, about 6.6 wt %, about 6.7 wt %, about 6.8 wt %, about 6.9 wt %, about 7.0 wt %, about 7.1 wt %, about 7.2 wt %, about 7.3 wt %, about 7.4 wt %, about 7.5 wt %, about 7.6 wt %, about 7.7 wt %, about 7.8 wt %, about 7.9 wt %, about 8.0 wt %, about 8.1 wt %, about 8.2 wt %, about 8.3 wt %, about 8.4 wt %, about 8.5 wt %, about 8.6 wt %, about 8.7 wt %, about 8.8 wt %, about 8.9 wt %, about 9.0 wt %, about 9.1 wt %, about 9.2 wt %, about 9.3 wt %, about 9.4 wt %, about 9.5 wt %, about 9.6 wt %, about 9.7 wt %, about 9.8 wt %, about 9.9 wt %, about 10 wt %, or any range including and/or in between any two of the preceding values.

Additionally or alternatively, in some embodiments of the three-dimensional biomimetic platform systems of the present technology, the collagen of the biocompatible substrate is a Type I collagen, a Type II collagen, a Type III collagen, a Type IV collagen, a Type V collagen, a Type VI collagen, a Type VII collagen, a Type VIII collagen, a Type IX collagen, a Type X collagen, a Type XI collagen, a Type XII collagen, a Type XIII collagen, a Type XIV collagen, a Type XV collagen, a Type XVI collagen, a Type XVII collagen, a Type XVIII collagen, a Type XIX collagen, a Type XX collagen, a Type XXI collagen, a Type XXII collagen, a Type XXIII collagen, a Type XXIV collagen, a Type XXV collagen, a Type XXVI collagen, a Type XXVII collagen, a Type XXVIII collagen, a Type XXIX collagen, or any mixture thereof. Type I collagen, Type II collagen, Type III collagen, Type IV collagen, and Type V collagen are the five most abundant types of collagen found in animals, whereas Type VI-Type XXIX collagens are rare.

In any of the embodiments of the three-dimensional biomimetic platform systems disclosed herein, the ratio of the Type I collagen to Type II collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios. In any of the embodiments of the three-dimensional biomimetic platform systems disclosed herein, the ratio of the Type I collagen to Type III collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios. In any of the embodiments of the three-dimensional biomimetic platform systems disclosed herein, the ratio of the Type I collagen to Type IV collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios. In any of the embodiments of the three-dimensional biomimetic platform systems disclosed herein, the ratio of the Type I collagen to Type V collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios.

In any of the embodiments of the three-dimensional biomimetic platform systems disclosed herein, the ratio of the Type II collagen to Type III collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios. In any of the embodiments of the three-dimensional biomimetic platform systems disclosed herein, the ratio of the Type II collagen to Type IV collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios. In any of the embodiments of the three-dimensional biomimetic platform systems disclosed herein, the ratio of the Type II collagen to Type V collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios.

In any of the embodiments of the three-dimensional biomimetic platform systems disclosed herein, the ratio of the Type III collagen to Type IV collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios. In any of the embodiments of the three-dimensional biomimetic platform systems disclosed herein, the ratio of the Type III collagen to Type V collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios.

In any of the embodiments of the three-dimensional biomimetic platform systems disclosed herein, the ratio of the Type IV collagen to Type V collagen is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios.

In any of the embodiments of the three-dimensional biomimetic platform systems disclosed herein, the ratio of abundant collagen (e.g., Type I collagen, Type II collagen, Type III collagen, Type IV collagen, Type V collagen, or any combination thereof) to rare collagen (e.g., Type VI collagen, Type VII collagen, Type VIII collagen, Type IX collagen, Type X collagen, Type XI collagen, Type XII collagen, Type XIII collagen, Type XIV collagen, Type XV collagen, Type XVI collagen, Type XVII collagen, Type XVIII collagen, Type XIX collagen, Type XX collagen, Type XXI collagen, Type XXII collagen, Type XXIII collagen, Type XXIV collagen, Type XXV collagen, Type XXVI collagen, Type XXVII collagen, Type XXVIII collagen, Type XXIX collagen, or any combination thereof) is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or any range or subrange between any two of the preceding ratios.

Additionally or alternatively, in some embodiments of the three-dimensional biomimetic platform systems of the present technology, the biocompatible substrate further comprises at least one non-collagen extracellular matrix component selected from the group consisting of fibronectin, laminin, hyaluronic acid, Matrix-bound nanovesicles (MBVs), elastin, proteoglycans, glycosaminoglycans (GAGs), heparan sulfate, perlecan, agrin, chondroitin sulfate, and keratan sulfate.

In any of the embodiments of the three-dimensional biomimetic platform systems disclosed herein, the ratio of collagen to the at least one non-collagen extracellular matrix component is about 99.99:0.01 or about 1:99. For example, in some embodiments, the ratio of collagen to the at least one non-collagen extracellular matrix component is about 99.99:0.01, about 99.9:0.1, about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, about 1:99, or any range or subrange between any two of the preceding ratios.

Additionally or alternatively, in some embodiments of the three-dimensional biomimetic platform systems of the present technology, the biocompatible substrate comprises about 25×10⁶ adipocytes per 2.6×10⁶ SVF cells, about 45,000 adipocytes per 4,680 SVF cells, or about 3×10⁶ adipocytes per 312,000 SVF cells. In some embodiments, the biocompatible substrate comprises an adipocyte to SVF cell ratio of about 10.5:1, about 10.4:1, about 10.3:1, about 10.2:1, about 10.1:1, about 10:1, about 9.9:1, about 9.8:1, about 9.7:1, about 9.6:1, about 9.5:1, about 9.4:1, about 9.3:1, about 9.2:1, about 9.1:1, about 9:1, or any range or subrange between any two of the preceding ratios. As used herein, “v/v adipocytes” refers to the volume of the adipocytes+SVF over the total volume of the biocompatible substrate present in the biomimetic platform system described herein. In some embodiments, the biocompatible substrate comprises about 5% v/v, about 6% v/v, about 7% v/v, about 8% v/v, about 9% v/v, about 10% v/v, about 11% v/v, about 12% v/v, about 13% v/v, about 14% v/v, about 15% v/v, about 16% v/v, about 17% v/v, about 18% v/v, about 19% v/v, about 20% v/v, about 21% v/v, about 22% v/v, about 23% v/v, about 24% v/v, about 25% v/v, about 26% v/v, about 27% v/v, about 28% v/v, about 29% v/v, about 30% v/v, about 31% v/v, about 32% v/v, about 33% v/v, about 34% v/v, about 35% v/v, about 36% v/v, about 37% v/v, about 38% v/v, about 39% v/v, about 40% v/v, or any range including and/or in between any two of the preceding values.

Additionally or alternatively, in some embodiments of the three-dimensional biomimetic platform systems of the present technology, the biocompatible substrate comprises an amount of patient-specific cells that ranges from about 50,000 cells to about 3.5×10⁶ cells. In some embodiments, the biocompatible substrate comprises about 5×10⁴ cells, about 5.5×10⁴ cells, about 6×10⁴ cells, about 6.5×10⁴ cells, about 7×10⁴ cells, about 7.5×10⁴ cells, about 8×10⁴ cells, about 8.5×10⁴ cells, about 9×10⁴ cells, about 9.5×10⁴ cells, about 1×10⁵ cells, about 1.5×10⁵ cells, about 2×10⁵ cells, about 2.5×10⁵ cells, about 3×10⁵ cells, about 3.5×10⁵ cells, about 4×10⁵ cells, about 4.5×10⁵ cells, about 5×10⁵ cells, about 5.5×10⁵ cells, about 6×10⁵ cells, about 6.5×10⁵ cells, about 7×10⁵ cells, about 7.5×10⁵ cells, about 8×10⁵ cells, about 8.5×10⁵ cells, about 9×10⁵ cells, about 9.5×10⁵ cells, about 1×10⁶ cells, about 1.5×10⁶ cells, about 2×10⁶ cells, about 2.5×10⁶ cells, about 3×10⁶ cells, about 3.5×10⁶ cells, or any range including and/or in between any two of the preceding values.

In any of the embodiments of the three-dimensional biomimetic platform systems disclosed herein, the one or more conduits have a shape selected from the group consisting of straight, curved, U-shape, zigzagged or any combination thereof that is suitable for culturing cells. In certain embodiments, the diameter of the one or more conduits ranges from about 100 μm to about 10 mm. In certain embodiments, the diameter of the one or more conduits is about 100 μm, about 200 μm, about 300 μm, about 400 μm, about 500 μm, about 600 μm, about 700 μm, about 800 μm, about 900 μm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, or any range including and/or in between any two of the preceding values. Additionally or alternatively, in some embodiments, the volume of the one or more conduits may range from about 100 μL to about 3 mL. In certain embodiments, the diameter of the one or more conduits is about 100 μL, about 200 μL, about 300 μL, about 400 μL, about 500 μL, about 600 μL, about 700 μL, about 800 μL, about 900 μL, about 1 mL, about 1.5 mL, about 2 mL, about 2.5 mL, about 3 mL, or any range including and/or in between any two of the preceding values. The diameter and/or volume of each conduit may be identical or distinct. Additionally or alternatively, in some embodiments, the walls of the one or more conduits may be smooth, have ridges, or may have a combination of smooth and ridged areas.

The one or more conduits may be uniform or non-uniform. Additionally or alternatively, in some embodiments, the one or more conduits are parallel to each other or intersect with each other to form a network. In certain embodiments, the network may be a hierarchal structure comprising conduits of variable length and diameter. The network can be an independent network with conduits forming an intersection with another conduit, a loop, a dead end or an open end. Additionally or alternatively, in some embodiments, the network may connect to or become continuous with an external network, such as a subject's circulatory system or another biomimetic platform system. Such continuity may be achieved though anastomosis of an open-end conduit. Additionally or alternatively, in some embodiments, the one or more conduits may arborize and/or coalesce into a vascular network. Additionally or alternatively, in some embodiments, a hierarchal network seeded with patient-specific cells may mature into vessels, vascular channels and ducts that are cellularized with a full complement of patient-specific cells characteristic for a particular subject's tissue or organ and may be surrounded by native extracellular matrix including the proteins and cells specific to the particular tissue or organ. For example, all vascular cell types are cultured to recapitulate a vascular network, or adipocytes and their supporting cells are cultured to recapitulate an adipose tissue.

Additionally or alternatively, in some embodiments, the three-dimensional biomimetic platform systems may further include a perfusion liquid or gas. For example, the biomimetic platform system may be perfused with a liquid or gas using an automated or manually operated pump. In some embodiments, the biomimetic platform systems can create anatomically and mechanically tunable, fully cellularized living tissue constructs, with vascular and lymphatic microvessel networks that can be perfused with pumps, along with concurrent epithelialized ducts.

### Methods for Fabricating the Biomimetic Platform Systems of the Present Technology

In one aspect, the present disclosure provides a method for producing a biomimetic platform of the present technology comprising (a) preparing a biocompatible substrate comprising a stromal vascular fraction, adipocytes, organoids, and at least one collagen, (b) adding patient-specific cells to the biocompatible substrate, and (c) culturing the patient-specific cells under conditions that permit maturation of the patient-specific cells in the biocompatible substrate. The patient-specific cells may be homogenously or heterogeneously within the biocompatible substrate.

In another aspect, the present disclosure provides a method for producing a biomimetic platform system of the present technology comprising (a) preparing a biocompatible substrate, (b) embedding a sacrificial material within the biocompatible substrate, (c) degrading the sacrificial material to produce one or more conduits within the biocompatible substrate, and (d) applying patient-specific cells to the one or more conduits within the biocompatible substrate. In some embodiments, the method further comprises culturing the patient-specific cells under conditions that permit maturation of the patient-specific cells in the biocompatible substrate. The biocompatible substrate may be any polymer suitable for culturing cells, providing a medium for the cells to attach to or providing a suitable environment for a cell suspension. Additionally or alternatively, in certain embodiments, the biocompatible substrate further comprises one or more components selected from the group consisting of stromal vascular fraction, adipocytes, and organoids.

Additionally or alternatively, in some embodiments of the methods disclosed herein, the stromal vascular fraction, adipocytes, and/or organoids are isolated by digesting a tissue sample with collagenase Type I and/or hyaluronidase.

In any of the above embodiments of the methods disclosed herein, the biocompatible substrate comprises at least one collagen (e.g., Type I collagen, Type II collagen, Type III collagen, Type IV collagen, Type V collagen). The at least one collagen may be neutralized with HEPES buffer or NaOH prior to embedding the sacrificial material within the biocompatible substrate. Additionally or alternatively, the at least one collagen may be modified with a glycosylating agent (e.g., glucose, ribose, fructose, galactose, glucose-6-Phosphate etc.) to modulate the stiffness of the biocompatible substrate.

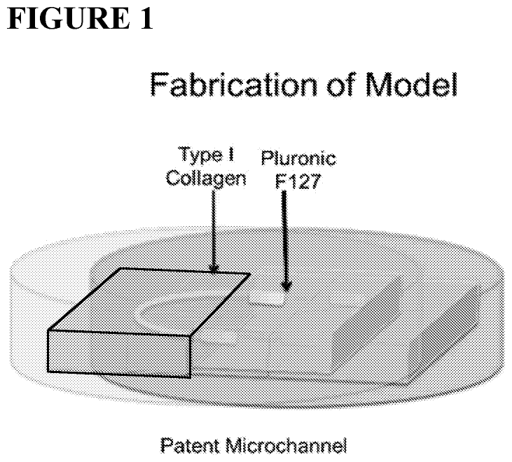

The sacrificial material may be any polymer that is capable of being degraded by manipulating physical characteristics of surrounding environment such as temperature. Examples of suitable sacrificial materials include, but are not limited to, poloxamers, shellac, carbohydrate glass, polyvinyl alcohol (PVA), and gelatin microparticles. Examples of poloxamers include, but are not limited to poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 Benzoate, and poloxamer 182 dibenzoate. In some embodiments, the sacrificial material is poloxamer 407 (e.g., Pluronic® F127). See FIG. 1.

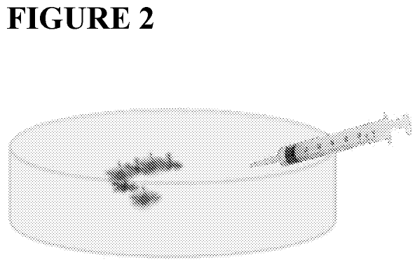

Additionally or alternatively, in some embodiments, the methods include identifying a subject in need of a biomimetic platform system disclosed herein and harvesting the patient-specific cells from the subject. The biomimetic platform system disclosed herein is useful for mimicking one or more organs or tissues of the subject. The patient-specific cells may be applied to the one or more conduits (e.g., seeded) with a syringe (see, e.g., FIG. 2). The biomechanical properties of the biocompatible substrate surrounding the one or more conduits seeded with the patient-specific cells may closely mimic the subject's extracellular matrix, stromal microenvironment and unique characteristics of organs and tissues. Examples of patient-specific cells include, but are not limited to, cancerous cells, pre-cancerous cells, pericytes, stem cells, blood cells, immune cells, platelets, central nervous system neurons, glial cells, peripheral nervous system neurons, skeletal muscle cells, smooth muscle cells, chondrocytes, bone cells, skin cells, hepatic cells, endothelial cells, epithelial cells, cardiac cells, pancreatic cells, adipocytes, gastric cells, intestinal cells, renal cells, fibroblasts, gall bladder cells, duct cells, pneumocytes, lens cells, sensory transducer cells, autonomic neurons, gland cells, hormone secreting cells, nurse cells, germ cells, or any combination thereof.

Additionally or alternatively, in some embodiments, the methods further comprise adding one or more biomolecules to the biocompatible substrate to promote cell culture and cell viability (e.g., growth factors, blood, plasma, hormones, cytokines, enzymes, vitamins, fatty acids, lymphokines, and the like). Examples of such biomolecules include, but are not limited to, angiopoietin, bone morphogenetic proteins (BMPs), ciliary neurotrophic factor (CNTF), leukemia inhibitory factor (LIF), macrophage colony-stimulating factor (m-CSF), granulocyte colony-stimulating factor (G-CSF), granulocyte macrophage colony-stimulating factor (GM-CSF), epidermal growth factor (EGF), ephrins, erythropoietin (EPO), fibroblast growth factors (FGF), glial cell line-derived neurotrophic factor (GDNF), neurturin, persephin, artemin, growth differentiation factor-9 (GDF9), hepatocyte growth factor (HGF), hepatoma-derived growth factor (HDGF), insulin-like growth factor-1 (IGF-1), insulin-like growth factor-2 (IGF-2), keratinocyte growth factor (KGF), migration-stimulating factor (MSF), hepatocyte growth factor-like protein (HGFLP), myostatin, neuregulins, brain-derived neurotrophic factor (BDNF), nerve growth factor (NGF), neurotrophin-3 (NT-3), neurotrophin-4 (NT-4), placental growth factor (PGF), platelet-derived growth factor (PDGF), Renalase (RNLS), T-cell growth factor (TCGF), thrombopoietin (TPO), transforming growth factor alpha (TGF-α), transforming growth factor beta (TGF-β), tumor necrosis factor-alpha (TNF-α), vascular endothelial growth factor (VEGF), Wnt ligands, Fetal Bovine Somatotrophin (FBS), Interleukin-6 (IL-6), insulin, interferon, IL-1, IL-2, IL-3, IL-4, IL-5, IL-6, IL-7, human growth hormone (hGH) etc.

### Uses of the Biomimetic Platform Systems of the Present Technology

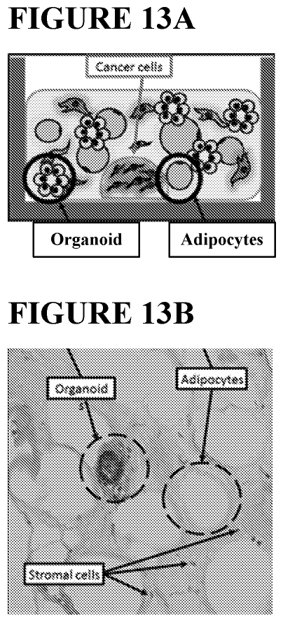

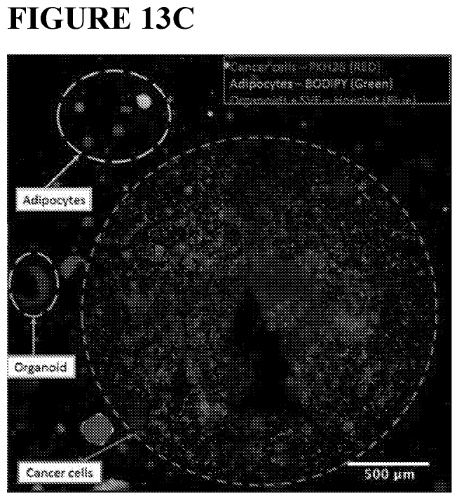

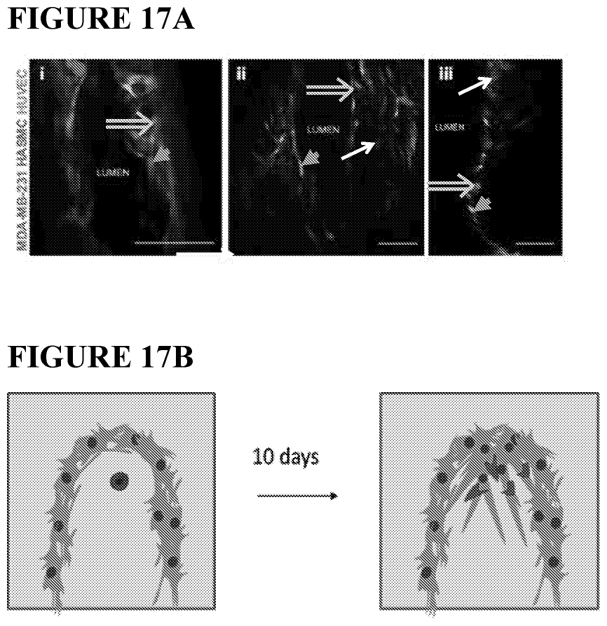

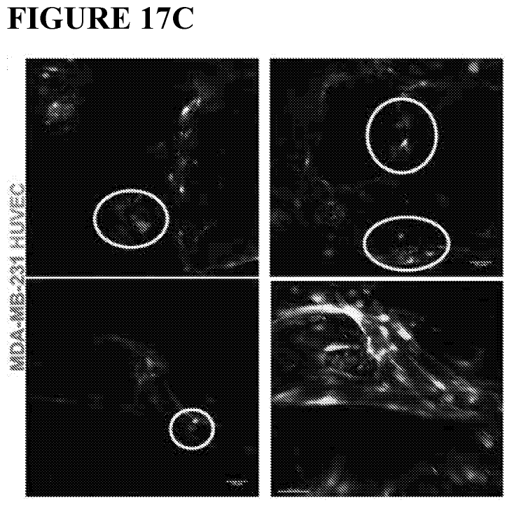

The biomimetic platform systems of the present technology may be used for diagnostics, drug screening, including timed release of drug and toxicity studies, as well as other biomedical research. For example, for breast cancer drug screening, the breast epithelial and myoepithelial cells of a subject may be used to line fabricated breast ducts; endothelial cells, smooth muscle cells and pericytes may be used to establish a vascularized network, lymphatic endothelial cells may be used to establish lymphatic channels; and fibroblasts, adipose derived stem cells and adipocytes may be seeded into the surrounding extracellular matrix recapitulated by the biocompatible substrate as shown in FIGS. 13A-13C, and 17B.

In one aspect, the present disclosure provides a method for monitoring at least one biological activity of patient-specific cells ex vivo comprising (a) culturing patient-specific cells in a biomimetic platform system of the present technology under conditions that permit maturation of the patient-specific cells; and (b) assaying at least one biological activity of the patient-specific cells. Additionally or alternatively, in some embodiments, the method further comprises implanting mature patient-specific cells from the biomimetic platform system into a host organism (e.g., a rodent such as a mouse or a rat). In certain embodiments, the implanted mature patient-specific cells are anastomosed to and perfused by the circulatory system of the host organism. Examples of suitable biological activities include, but are not limited to cell viability, cell growth, cell division, apoptosis, cell migration, angiogenesis, gene expression, blood coagulation, metastasis etc. The patient-specific cells may comprise any one or more cell types disclosed herein.

In one aspect, the present disclosure provides a method for screening the effect of a candidate agent on patient-specific cells comprising (a) contacting the candidate agent with a biomimetic platform system of the present technology, wherein the biomimetic platform system comprises patient-specific cells that are cultured under conditions that permit maturation of the patient-specific cells, and (b) assaying at least one biological activity of the treated patient-specific cells. In some embodiments, the treated patient-specific cells exhibit an alteration in at least one biological activity compared to that observed in untreated patient-specific cells. Examples of suitable biological activities include, but are not limited to cell viability, cell growth, cell division, apoptosis, cell migration, angiogenesis, gene expression, blood coagulation, metastasis etc. The patient-specific cells may comprise any one or more cell types disclosed herein.

In another aspect, the present disclosure provides a method for evaluating the toxicity of a candidate agent on patient-specific cells obtained from a healthy subject comprising (a) contacting the candidate agent with a biomimetic platform system of the present technology, wherein the biomimetic platform system comprises patient-specific cells that are cultured under conditions that permit maturation of the patient-specific cells, (b) assaying the viability of the treated patient-specific cells, and (c) determining that the candidate agent is toxic when the treated patient-specific cells exhibit decreased viability compared to that observed in untreated patient-specific cells.

In one aspect, the present disclosure provides a method for determining the therapeutic efficacy of a candidate agent for treating a disease (e.g., cancer) in a patient in need thereof comprising (a) contacting a biomimetic platform system of the present technology with the candidate agent, wherein the biomimetic platform system comprises patient-specific diseased cells that are cultured under conditions that permit maturation of the patient-specific diseased cells, and (b) determining that the candidate agent is therapeutically effective when the treated patient-specific diseased cells exhibit decreased viability compared to that observed in untreated patient-specific cells.

The candidate agent may be a synthetic low-molecular-weight compound, a natural compound, a recombinant protein, a purified or crude protein, a peptide, a non-peptide compound, an antibody, an engineered cell, a vaccine, a nucleic acid (e.g., a siRNA, an antisense oligonucleotide, a sgRNA, an aptamer), a recombinant virus, a recombinant microorganism, a ribozyme, a cell extract, a cell culture supernatant, a microbial fermentation product, a marine organism extract, a plant extract, or any combination thereof. In some embodiments, the candidate agent is a chemotherapeutic agent. Examples of chemotherapeutic agents include 5-FU, nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicin, doxorubicin analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11. For high-throughput screening methods, the effects of individual candidate agents may be assessed using microwell arrays. The microwells may be sealed by mechanical sealing, oil sealing, or by another means. In some embodiments, alterations in biological activities may be detected via microscopy, scanning, or other imaging assays.

Additionally or alternatively, in some embodiments of the methods disclosed herein, the patient-specific cells are isolated from a healthy subject or a subject that has been diagnosed with or is suffering from a disease. In some embodiments, the subject is human. In certain embodiments, the disease is a cancer selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

In some embodiments, the biomimetic platform system of the present technology may be utilized for high throughput analysis of patient-specific tumor behavior. The biomimetic platform system may enable rapid and flexible biochemical, genomic, metabolic analysis or any combination of analysis thereof using a wide variety of standard assays, such as immunohistochemistry, Western Blot analysis, fluorescence microscopy, FACS analysis, TUNEL analysis, H&E staining, RNA-Seq, ATAC-Seq, or any other existing technologies known in the art (See e.g., FIGS. 17A and 17C).

### EXAMPLES

The present technology is further illustrated by the following Examples, which should not be construed as limiting in any way.

### Example 1: Materials and Methods for Assembling the Biomimetic Platform Systems of the Present Technology

Collagen Extraction.

Tendons excised from commercially available rat tails were manually dissected and suspended in 0.1% acetic acid using 75 mL of acid per gram of tendons in order to extract type I collagen. Following complete dissolution of tendons in acetic acid (72 hours at 4° C.), the solution was ultra-centrifuged at 8,800×g for 2 hours to remove remaining tissue debris. Supernatant was collected, frozen and lyophilized. Lyophilized product was then resuspended in 0.1% acetic acid to form a working solution of 15 mg/mL.

Collagen Neutralization.

Purified type I collagen extracted from rat tails and dissolved in 0.1% acetic acid at a starting concentration of 15 mg/mL was neutralized with 1M NaOH and diluted with M199 media to achieve a working concentration of 6 mg/mL. Neutralized collagen was kept at 4° C. to prevent nucleation until cellular components were added and collagen/cell mix was delivered to the desired mold. Once neutralized collagen was ready for nucleation, molds containing either collagen only or a collagen/cell mixture were allowed to nucleate for 30 minutes at 37° C. After nucleation was achieved, constructs were submerged in the cell culture media corresponding to the cell types utilized.

Fluorescent Labeling of Cell Lines for Use in Constructs.

Lentivirus transfected human cell lines were selected for use in the biomimetic platform systems disclosed herein. Color selection was randomly assigned to ensure proper differentiation between cell types under fluorescent and confocal microscopy. For vascularized constructs, Human Umbilical Vein Endothelial Cells (HUVECs) were transfected with green fluorescent protein (GFP), Human Aorta Smooth Muscle Cells (HASMCs) were labeled with mCherry, and Human Placental Pericytes (HPLPs) were tagged with cyan fluorescent protein. Cancer cell lines, MDA-MB231, MDA-MB468, and HS-578T were transfected with mCherry lentivirus.

Isolation of Patient-Tissue Components.

Surgical specimens were obtained. In a sterile biosafety hood, breast specimens were homogeneously minced. Excess lipid was carefully removed from tissue by suction, and minced tissue was mixed on a 1:1 ratio with complete Ham's media containing 1 mg/mL of collagenase Type I and 0.01 mg/mL hyaluronidase (for adipocytes and Adipose derived stem cell (ASC)-containing stromal vascular fraction (SVF) isolation) or 1 mg/mL of collagenase type IA and 0.01 mg/mL hyaluronidase (for isolation of breast organoids).

Adipocyte and SVF digestion was performed by placing a 50 mL conical tube containing 1:1 mixture of minced tissue and collagenase Type I with hyaluronidase, in a pre-warmed shaker incubator for 1 hour at 37° C. After digestion, the cell preparation was centrifuged at 800×g for 10 minutes. Following centrifugation, mature adipocytes were collected using wide-bore micropipette tips and mixed with equal volume of warm complete Ham's media with 10% FBS and 1% Penicillin/Streptomycin, mixed by inverting and allowed to separate.

Digested tissue components remaining in conical tube after collection of adipocytes were used for SVF isolation. The media/collagenase interphase was discarded to preserve only the pellet which was incubated at room temperature for 10 minutes in RBC lysis buffer after sequential filtering through 100 μM and 40 μM cell strainers. Following incubation with RBC lysis buffer, SVF was pelleted by centrifugation, and subsequently reconstituted in DMEM/F12 with 10% FBS and 1% Penicillin/Streptomycin.

Organoid isolation was performed by placing a 50 mL conical tube containing 1:1 mixture of minced tissue and collagenase Type I with hyaluronidase, in a pre-warmed shaker incubator for 3 hours at 37° C. After digestion, the cell preparation was centrifuged at 800×g for 10 minutes, followed by discarding of supernatant. The remaining pellet was then dissolved in DMEM/F12 and placed for 30 minutes at 4° C. to neutralize collagenase and subsequently centrifuged at 800×g for 10 minutes. Pellet was resuspended in RBC lysis buffer and incubated at room temperature for 10 minutes on a rocking platform to ensure occasional mixing of mixture. After RBC lysis buffer incubation, the tube was centrifuged at 800×g for 10 minutes to form a pellet. The supernatant was discarded and the remaining pellet was reconstituted in DMEM 1×, and filtered through 100 and 40 μm cell strainers. The filtrate was discarded. The organoid-containing fraction that remained attached to strainers was collected and reconstituted in Mammary Epithelial Cell Growth Media (MEGM) with growth supplements and 1% Penicillin/Streptomycin.

Staining of Mature Adipocytes.

BODIPY (493/503) (Invitrogen, ThermoFisher Scientific™, Waltham, Mass., US) was used at a concentration of 1 μg/mL to stain lipids contained within mature, isolated adipocytes. Incubation was performed for 30 minutes at 37° C. Following incubation, the stained adipocytes were washed with DMEM/F12 (10% FBS 1% Penicillin/Streptomycin) and maintained away from direct light.

Assembly of Collagen Only and Ex-Vivo Breast Biomimetic Stroma with Adipocytes and Cancer Cells.

The total amount of 1×M199 media needed for collagen dilution during the neutralization process was reduced by 2504, to allow for the volume needed for reconstitution of cellular components that were added to neutralize collagen. The Collagen platform without breast components was fully diluted by adding 250 microliters of media with desired number of vascular cells (e.g., pericytes), while the biomimetic platform with breast components was diluted by adding 250 μL of MEGM media containing extracted organoids, SVF containing ASCs, and cancer cells. Components were mixed homogeneously into the neutralized collagen, and mature adipocytes were added to the biomimetic platform containing breast stroma at 15% v/v (v/v refers to the volume of the adipocytes+SVF over the total volume of the biocompatible substrate present in the biomimetic platform system described herein).

Sacrificial Macrofiber and Microfiber Fabrication.

A negative 1.5 mm diameter “U” shaped pattern was created within a PDMS mold. A sacrificial polymer, Pluronic® F127 (Sigma Aldrich®, St. Louis, Mo.) was warmed up to 70° C. and poured into Poly-dimethylsiloxane (PDMS, Slygard®, Dow Corning, Midland, Mich.) molds. After solidification at 4° C. for 10 minutes, the macrofibers were demolded.

Construct Fabrication.

Positive cylindrical molds were 3-D printed using polycarbonate to create different patterns. PDMS was poured into molds and cured for 30 mins at 80° C. PDMS molds were sterilized in thermal plasma cleaner and surface was activated by coating with glutaraldehyde. For U loop sacrificial macrofibers, adjacent 15 mm×15 mm×5 mm reservoirs connected by inlet and outlet channels were created on the same side of the reservoirs. Strategically placed fourteen-gauge catheters were introduced into inlet and outlet channels, and utilized to hold the sacrificial U loop. The PDMS and Pluronic® F127 loops were sterilized under Ultraviolet light for 24 hours prior to use. Alternatively, for the straight channel vascular model, a 15 mm×15 mm×5 mm reservoir with inlet and outlet channels on opposite walls of the reservoir, and a fourteen-gauge catheters were placed on each one of the channels. A needle of the same gauge was inserted through one channel and pushed through the opposite one, resulting in a naked needle suspended within the reservoir.

Under sterile conditions, the type I collagen based biomimetic platform was poured into molds and allowed to nucleate for 30 minutes at 37° C. as described above. During this process, the Pluronic® F127 loops were completely dissolved resulting in a channel within the constructs that was subsequently seeded with vascular cells. Alternatively, for the needle method for straight channel, the needle within the construct was carefully removed once nucleation of collagen was accomplished, resulting in a straight lumen within the platform. Nucleated constructs were submerged in a cell culture media mix consisting of equal parts MEGM, DMEM:F12, Endothelial Cell Growth Media, Smooth Muscle cell Growth media, and Pericyte growth media. Twenty-four hours following fiber sacrifice, a cell suspension of human aortic smooth muscle cells (HASMC) and human umbilical vein endothelial cells (HUVEC) was seeded into the main channel and allowed to develop the main vessel. After 7-10 days of culture, gels were fixed and processed for analysis.

Construct Fixing and Processing.

Biomimetic constructs were fixed in formaldehyde for 30 minutes followed by 3 consecutive 5-minute Phosphate Buffered Saline (PBS) washes. Finally a 1:1000 dilution of DAPI in PBS was used to replace the last PBS wash and samples were left at 4° C. overnight for DAPI penetration into cell nuclei. Images were analyzed by confocal microscopy.

Imaging of Biomimetic Platform Systems.

Biomimetic constructs were imaged using Zeiss LSM 880 Laser scanning confocal microscope at excitation and detection levels specific for the signals of interest. Specifically, DAPI and Hoechst signal was collected at 405-450 nm, BODIPY and GFP at 470-520 nm, and mCherry at 570-630 nm. Pericyte number and migration was assessed utilizing an upright Olympus FluoView FV1000MPE multiphoton microscope (Olympus America Inc. Center Valley, Pa., USA). Images were collected using three multi-alkali photomultiplier tubes (PMTs), each of which collected one of the signals of interest. Specifically, the CFP signal was collected at 420-460 nm, GFP at 495-540 nm, and RFP at 575-630 nm. Unseeded constructs with sacrificed networks were filled with 5 μm green fluorescent microspheres (Sigma Aldrich, St. Louis, Mo.) and imaged to illustrate patency of the channels.

Image Analysis and Quantification.

All multiphoton images acquired were analyzed either with Imaris™ (Bitplane South Windsor, Conn., USA) or Metamorph™ (Molecular Devices, Sunnyvale, Calif.). Metamorph™ was used for all image analysis and quantification of channel dimensions. Imaris™ was used for the visualization of the 3D image volume due to its inherent integration of automatic detection of objects in 3D space based on intensity and size, and associated ability to visualize complex structures, such as the hierarchical vascular network.

Confocal images for doxorubicin response were analyzed using Imaris™ for visualization of 3D image volume. Cancer cell counts were performed by defining cells and nuclei on the Cells function on Imaris™ software. Cells were filtered by presence of red cytoplasm, blue nuclei, size of cells, and nuclei, ellipticity, and signal intensity. The endothelial cell and pericyte fluorescence signals were visualized as a 3D volume. Each image channel was then individually thresholded in order to generate 3D surfaces. The surfaces were further filtered based on size criteria in order to reject small extraneous objects selected using intensity threshold alone. Distances of individual pericytes from their nearest endothelial cell neighbor were then measured, recorded, and exported to Microsoft Excel (Redmond, Wash.).

### Example 2: Tissue Culture Capabilities of the Biomimetic Platform Systems of the Present Technology

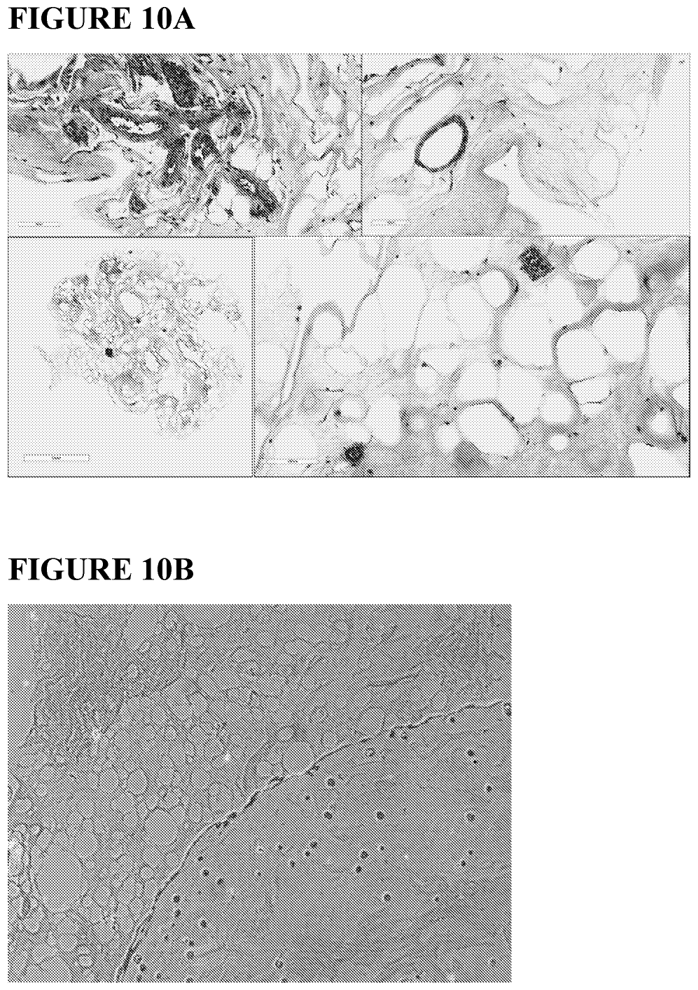

FIG. 10B shows a microscopic image of normal epithelial breast cells cultured with the 3D-breast component biomimetic platform system (0.6% collagen (w/v)+adipocytes, stromal cells, breast organoids). These results demonstrate that the ex vivo biomimetic platform systems of the present technology recapitulate the native in vivo environment and are useful in methods for assessing the toxicity of a candidate agent in biological sample obtained from a healthy control subject.

FIG. 17B is a schematic showing the placement of a cancer cell spheroid in an embodiment of the collagen only biomimetic platform system disclosed herein, and the progression of cancer metastasis in the mechanically tuned microenvironment created by the biomimetic platform system. FIG. 17C shows an example of a confocal micrograph of tumor-induced angiogenesis in a mechanically tuned (higher elastic compressive modulus) microenvironment (0.3% (w/v) collagen modified with 200 μM ribose and MDA-MB231 spheroid). After 10 days, neovessels (10-80 μm in diameter) formed towards the spheroid. MDA-MB231 cancer cells subsequently broke off and invaded the neovessels (circles identify locations of metastasis). FIG. 17A shows multiphoton microscopy images of (i) non-cancer containing constructs, and (ii)-(iii) cancer containing constructs with fluorescently tagged HUVEC, and HASMC cells. Cancer containing constructs show invasion of labelled MDA-MB231 cells towards the lumen of the neovessel, disrupting the endoluminal lining and sub adjacent smooth muscle cells. See FIG. 17(A) (ii)-(iii).

These results demonstrate that the ex vivo biomimetic platform systems of the present technology recapitulate the native in vivo environment and are useful in methods for monitoring cell behavior and/or metastasis of specific cells and tissues derived from a subject.

### Example 3: Shape of Conduits within the Biomimetic Platform Systems of the Present Technology Impacts Local Cell Behavior

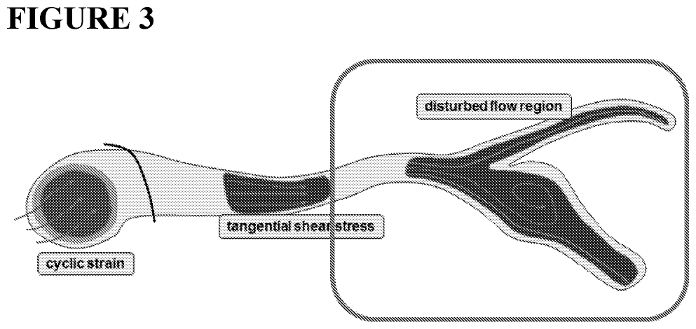

FIG. 3 shows a representation of the different types of flow that occur based on the shape and size of the conduits. Based on geometries and orientation of the conduits, cells encounter differential shear stresses. Hemodynamic shear stress can modulate endothelial cell behavior including angiogenic response and interactions between endothelial cells and mesenchymal cells. For instance, shear stress response by endothelial cells is mediated by junctional complexes that include VE cadherin, PECAM, and VEGFR2.

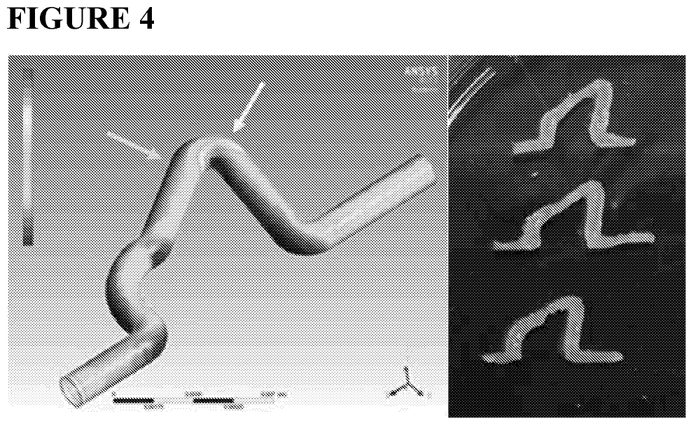

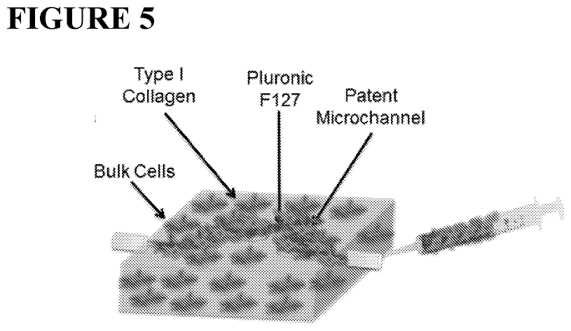

To assess the impact of conduit shape on cell behavior, a Pluronic® F127 microfiber with regions of different shear stresses was generated using the AnsysFluent fluid simulation software. See FIG. 4. Acute angles were predicted to provide regions of high shear stress, whereas linear regions resulted in regions of lower shear stress. A biomimetic platform comprising collagen bulked with pericytes was assembled. A conduit with the desired shape was generated in the biomimetic platform using the Pluronic® F127 sacrificial methods described in Example 1 and was seeded with smooth muscle cells and endothelial cells. See FIG. 5. The conduit facilitated the formation of a vascular structure in the biomimetic platform.

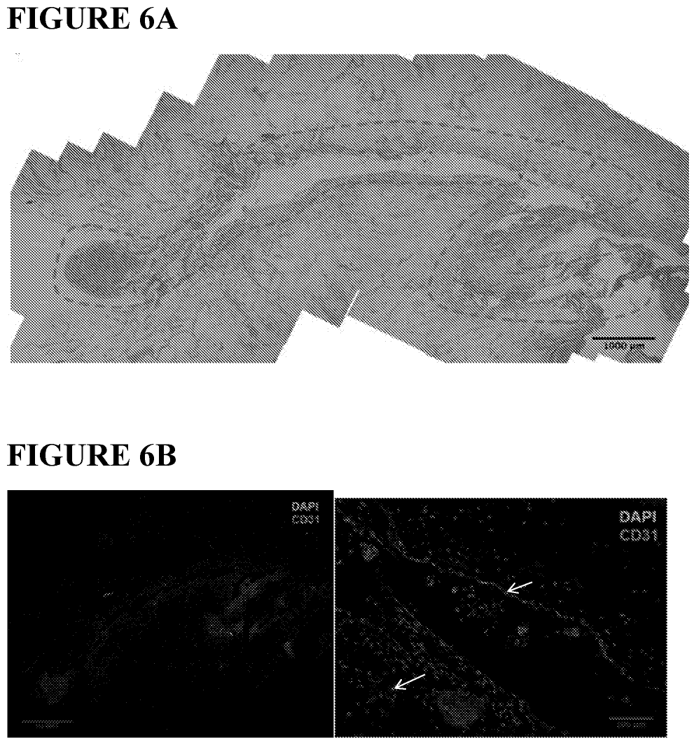

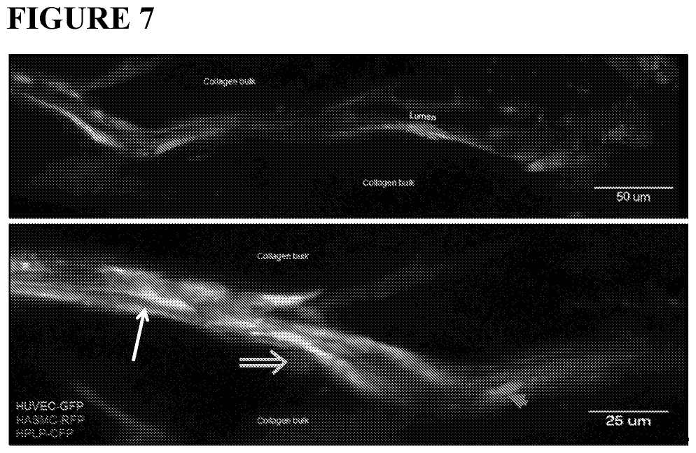

FIG. 6A shows the H&E staining of the vascular structure with lumen. Fluorescent microscopy revealed that CD31 positive endothelial cells lined the walls of the channel after 14 days of culture. See FIG. 6B.

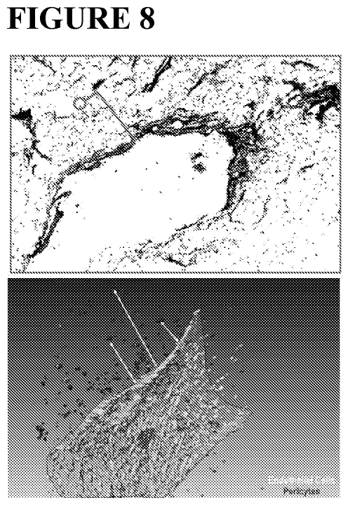

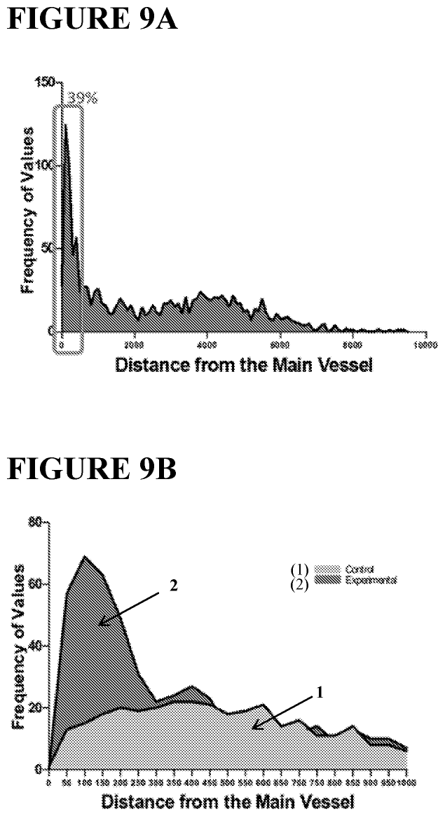

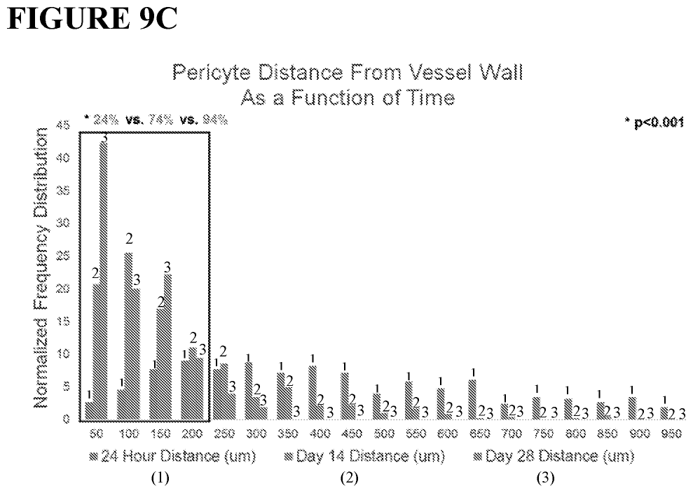

FIG. 8 shows the distribution of pericytes relative to the wall of the vessel/vascular structure (3D fluorescent microscopy model (right); black and white image rendition (left) of the same). The skewed frequency distribution presented in FIG. 9A demonstrates cellular migration towards the vessel, with 39% of cells being located within 500 μm of the newly formed vessel. As shown in FIG. 9B, the distribution of the cells in the vessel with shear stress was altered (e.g., increased cell numbers within close proximity to the vessel) compared to a control vessel with no induced shear stress. See also FIG. 9C showing the pericyte distance from the vessel wall as a function of time.

These results demonstrate that the ex vivo biomimetic platform systems of the present technology recapitulate the native in vivo environment and are useful in methods for culturing patient specific cells and tissues.

### Example 4: Effects of Collagen Density and Stiffness on the Biomimetic Platform Systems of the Present Technology

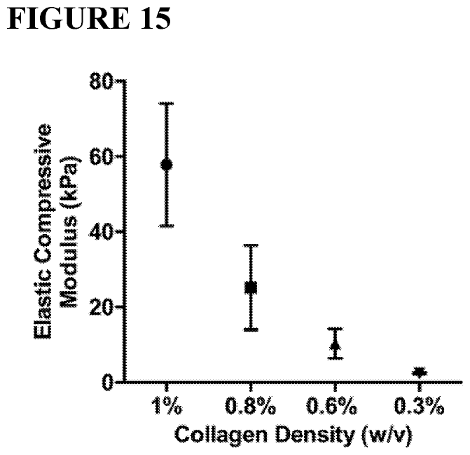

FIG. 15 shows the elastic compressive moduli of different densities of collagen gels. The density of the collagen gels impacted the elastic compressive modulus, with 1% collagen showing an elastic compressive modulus of about 60 kPa.

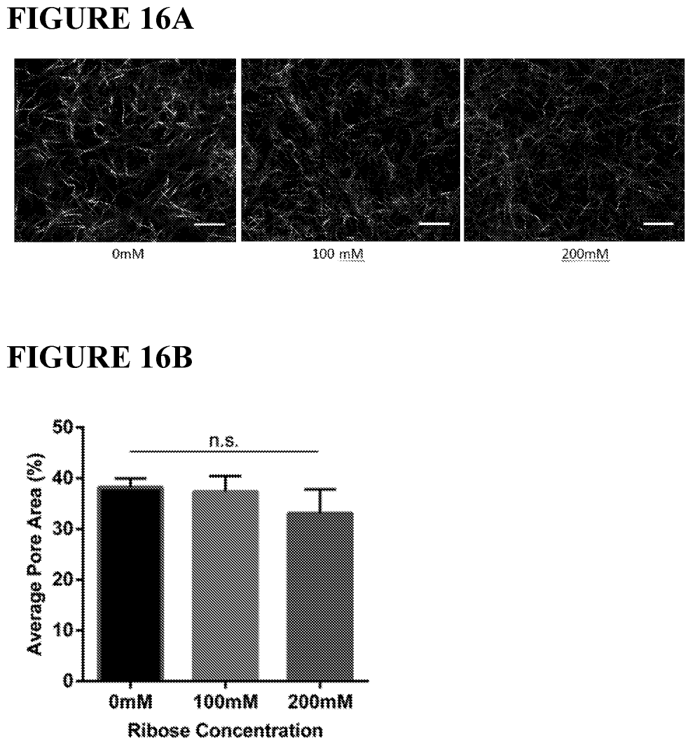

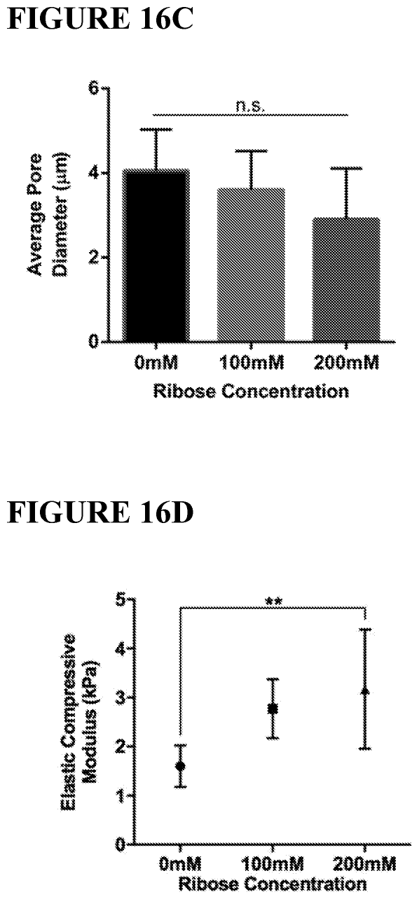

FIG. 16A shows the confocal reflectance microscopy results of 0.3% (w/v) collagen hydrogels when dosed with 0 mM, 100 mM, and 200 mM ribose solution. FIG. 16B shows the average pore area of 0.3% (w/v) collagen when dosed with 0 mM, 100 mM, and 200 mM ribose solution. FIG. 16C shows the average pore diameter of 0.3% (w/v) collagen when dosed with 0 mM, 100 mM, and 200 mM ribose solution. Taken together, these results demonstrate that the collagen only biomimetic platform system was not adversely affected when modified with a glycosylating agent.

FIG. 16D shows the elastic compressive moduli of 3 mg/ml collagen gels when dosed with 0 mM, 100 mM, and 200 mM ribose solution. The collagen gel showed a significant increase in elastic compressive modulus when treated with 200 mM ribose solution compared to that observed in a collagen gel that was not treated with a glycosylating agent.

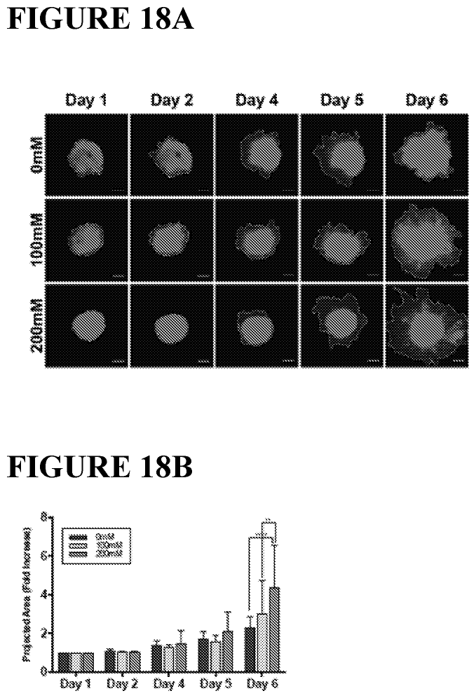

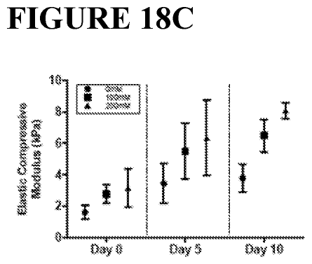

FIGS. 18(A)-18(C) show the spheroid cellular outgrowth of MDA-MB231 cancer cells in response to collagen stiffness. As shown in FIGS. 18(A)-18(B), MDA-MB231 cancer cells exhibited a significant increase in spheroid cellular outgrowth when cultivated in 0.3% (w/v) collagen gels dosed with 200 mM ribose solution (high stiffness) compared to that observed with collagen gels dosed with 0 mM or 100 mM ribose solution. FIG. 18(C) shows the increase in elastic compressive modulus of collagen gels dosed with 0 mM, 100 mM, and 200 mM ribose solution after incubation with MDA-MB231 spheroids over time. MDA-MB231 spheroid-containing collagen gels dosed with 200 mM ribose solution (high stiffness) exhibited the highest elastic compressive modulus at day 10 compared to that observed in MDA-MB231 spheroid-containing collagen gels dosed with 0 mM or 100 mM ribose solution.

These results demonstrate that the ex vivo biomimetic platform systems of the present technology recapitulate the native in vivo environment and are useful in methods for culturing patient specific cells and tissues.

### Example 5: Effects of Adipocyte Contents on the Biomimetic Platform Systems of the Present Technology

Adipocytes have been previously reported as playing a critical role in cancer progression and modifying tumor sensitivity to therapeutic agents. See Hoy A J et al., Trends Mol Med 23(5):381-392 (2017); Sheng X et al., Mol Cancer Res 15(12):1704-1713 (2017). Adipocytes have also previously been shown to take up chemotherapeutic agents and convert them to less active metabolites (Sheng X et al. (2017), supra). This Example demonstrates that the biomimetic platform systems of the present technology recapitulate this effect. Moreover, as shown in Example 6, breast cancer cells grown in the 3D-breast component biomimetic platform system (which includes adipocytes) were less sensitive to the effects of doxorubicin than those cultured in the 3D collagen only biomimetic platform system.

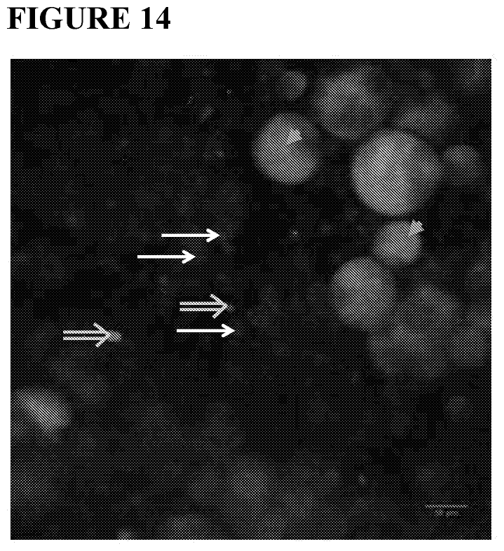

To recreate the tumor microenvironment, biomimetic platform systems including 0.6% (w/v) collagen, 25% v/v adipocytes, stromal cells, breast organoids, and cancer cells were assembled using the methods described in Example 1. FIG. 14 shows a confocal image of the biomimetic platform system including 25% v/v adipocytes. Cancer cells were visualized via Cytokeratin 19 staining. However, inclusion of 25% v/v adipocytes to the biomimetic platform system resulted in the inability to generate vascular channels with single or triple lumen.

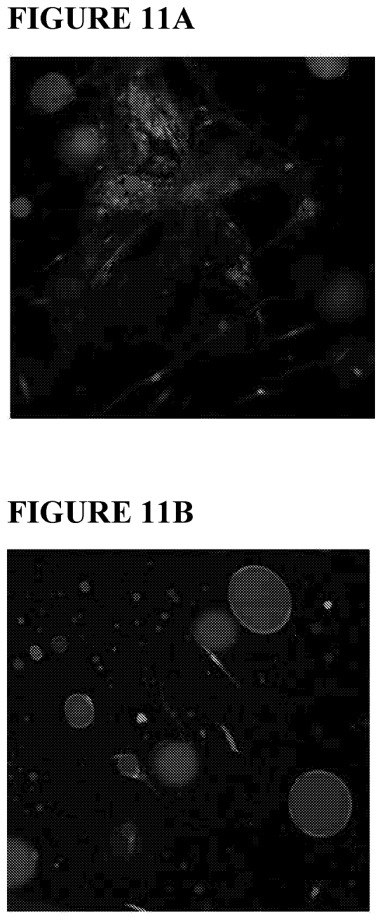

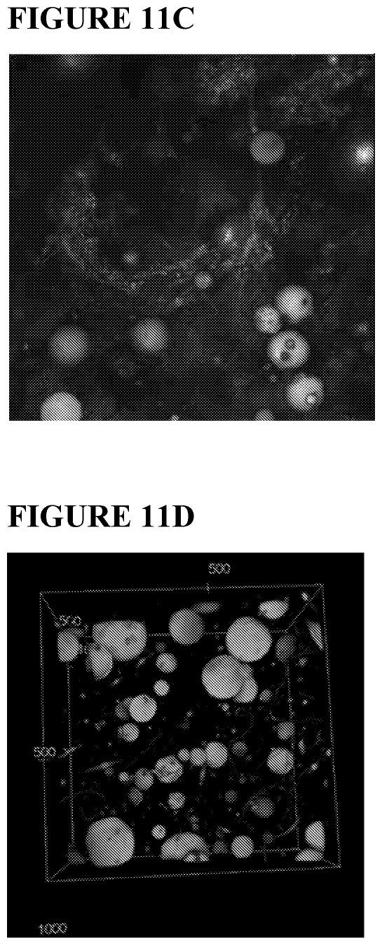

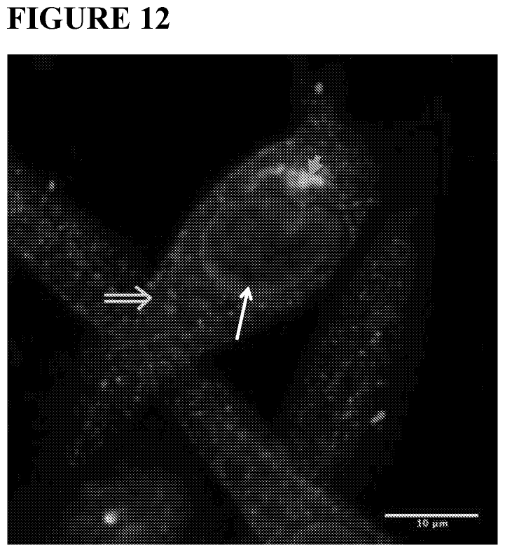

FIGS. 10A, 11A-11D, 12, 13A-13C are images of the biomimetic platform system including 15% v/v adipocytes. Vascular channels with single/triple lumen were successfully generated when 15% v/v adipocytes were utilized.

These results demonstrate that the ex vivo biomimetic platform systems of the present technology recapitulate the native in vivo environment and are useful in methods for culturing patient specific cells and tissues.

### Example 6: Tissue Permeability and Drug Screening Capabilities of the Biomimetic Platform Systems of the Present Technology

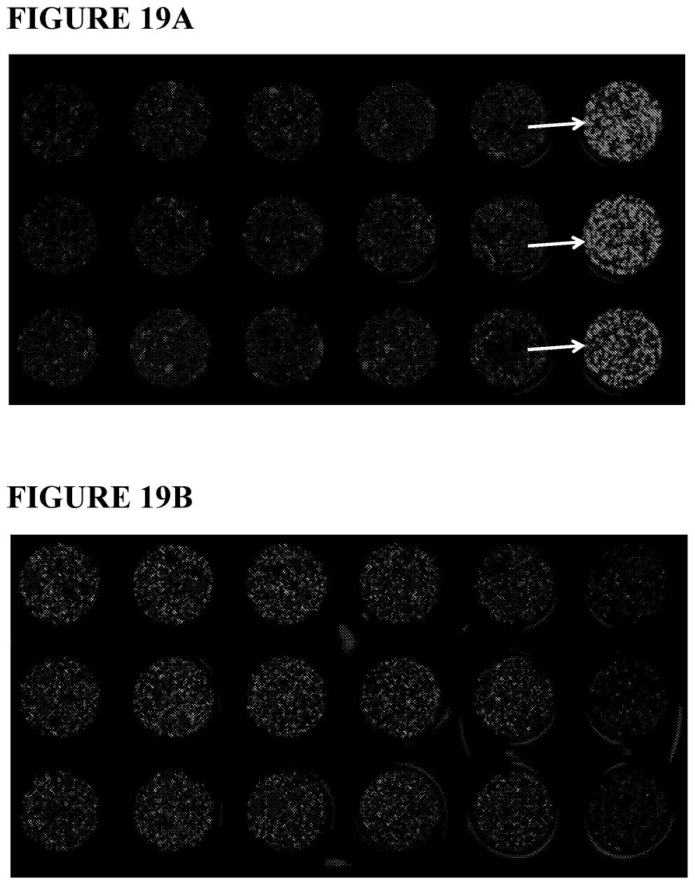

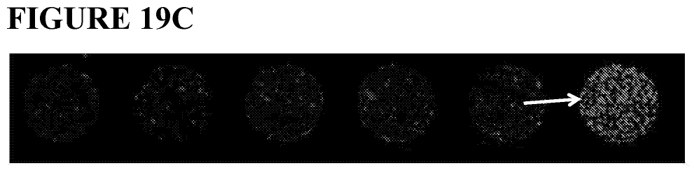

Biomimetic platform systems comprising 0.6% (w/v) collagen and 200,000 MDA-MB231 cancer cells labelled with mCherry were incubated with various concentrations of doxorubicin (0-10 μM). A decrease in the absolute number of cells (indicated by reduced mCherry and DAPI signals) was observed when the platforms were incubated with 1-10 μM doxorubicin, thus demonstrating the concentration-dependent cytotoxic effects of doxorubicin. See FIG. 19B.

Collagen gels (0.6% (w/v)) including 15% v/v adipocytes, stromal cells, and breast organoids, but without mCherry labelled MDA-MB231 cancer cells, were incubated with various concentrations of doxorubicin (0-10 μM). FIG. 19C demonstrates the permeability of the biomimetic platform and adipocytes to doxorubicin (as evidenced by increased signal) with increasing doxorubicin concentrations.

Collagen gels (0.6% (w/v)) including 15% v/v adipocytes, stromal cells, breast organoids, and 200,000 MDA-MB231 cancer cells labelled with mCherry were incubated with various concentrations of doxorubicin (0-10 μM). As shown in FIG. 19A, doxorubicin uptake was increased in biomimetic platform systems incubated with high concentrations of doxorubicin (1-10 μM). The large globules of doxorubicin signal observed in platforms on far right of FIG. 19A correspond with doxorubicin uptake by adipocytes. FIG. 19A demonstrates the permeability of the biomimetic platform and adipocytes to doxorubicin (as evidenced by increased signal) with increasing doxorubicin concentrations. A decrease in the absolute number of cells (indicated by reduced mCherry and DAPI signals) was also observed when the platforms were incubated with 1-10 μM doxorubicin, thus demonstrating the concentration-dependent cytotoxic effects of doxorubicin.

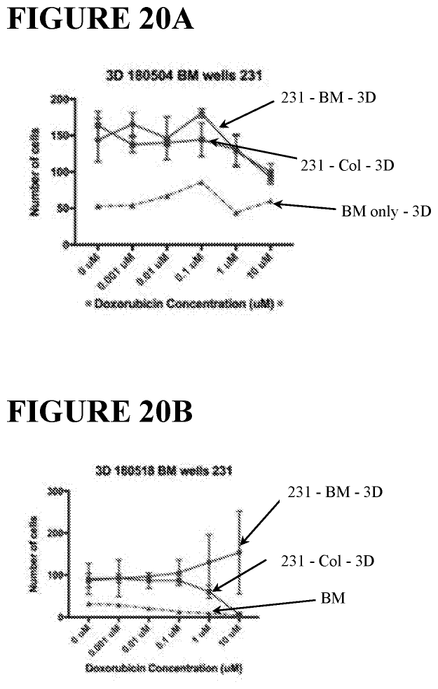

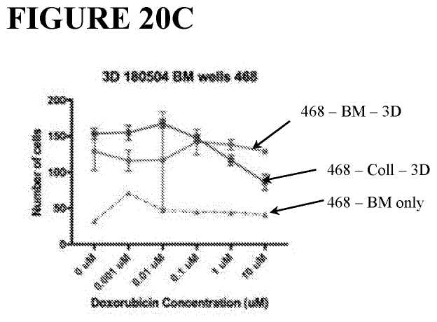

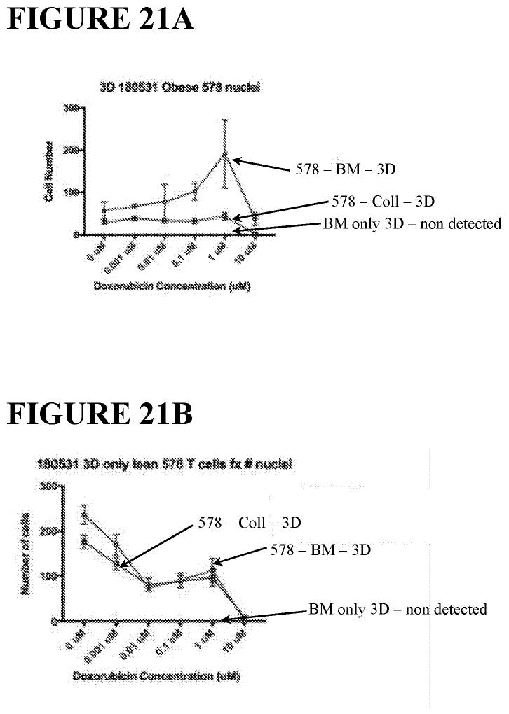

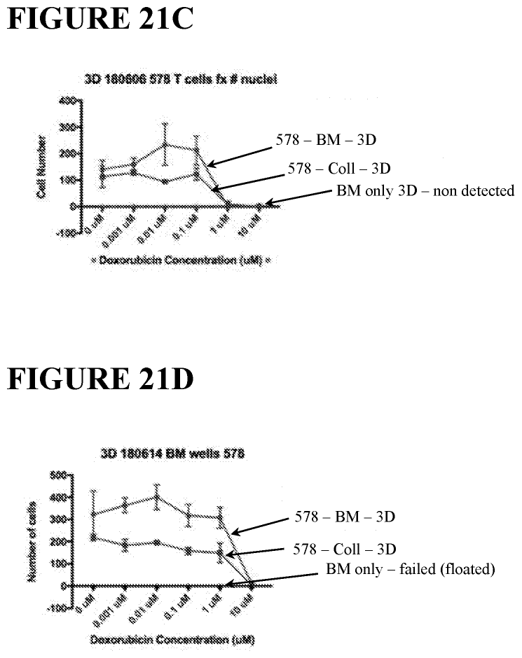

FIGS. 20A-20C, and 21A-21D compare the responsiveness of MDA-MB231, MDA-MB468, and HS-578T cancer cell lines to different concentrations of doxorubicin when cultured in the 3D-collagen only biomimetic platform system, the 3D-breast component biomimetic platform system comprising cancer cells, and the 3D-breast component biomimetic platform system without cancer cells (BM only). Doxorubicin exhibits intrinsic fluorescence, which is useful for tracking cellular uptake. Adipocytes have previously been shown to take up chemotherapeutic agents (Sheng X et al., Mol Cancer Res 15(12):1704-1713 (2017)). The biomimetic platform system disclosed herein successfully recapitulated this effect. Further, breast cancer cells cultured in the 3D-breast component biomimetic platform system were less sensitive to the effects of doxorubicin than those cultured in 3D-collagen only biomimetic platform system. Taken together, these results demonstrate that the ex vivo biomimetic platform systems of the present technology accurately recapitulate the 3D-tumor microenvironment and is thus useful for determining appropriate therapeutic agents as well as effective doses of the same for the treatment of cancer.

These results demonstrate that the ex vivo biomimetic platform systems of the present technology recapitulate the native in vivo environment and are useful in methods for determining an effective dose of a candidate agent for treating a disease (e.g., cancer) in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A three-dimensional biomimetic platform comprising
(a) a biocompatible substrate including collagen, a stromal vascular fraction, adipocytes, and organoids, and optionally lymphatic endothelial cells; and
(b) patient-specific cells, wherein the patient-specific cells are homogenously or heterogeneously dispersed within the biocompatible substrate.

2. A three-dimensional biomimetic platform system comprising
(a) a biocompatible substrate including collagen, wherein the biocompatible substrate comprises one or more conduits, optionally wherein the biocompatible substrate further comprises a stromal vascular fraction, adipocytes, and organoids and optionally lymphatic endothelial cells; and
(b) patient-specific cells cultured in the one or more conduits.

3. (canceled)

4. The three-dimensional biomimetic platform system of claim 2,
wherein the stromal vascular fraction comprises one or more of adipose-derived stem/stromal cells (ADSCs), endothelial precursor cells (EPCs), endothelial cells (ECs), macrophages, smooth muscle cells, lymphocytes, pericytes, and pre-adipocytes, and/or
wherein the organoids are breast organoids, cerebral organoids, intestinal organoids, gastric organoids, hepatic organoids, lingual organoids, thyroid organoids, thymic organoids, testicular organoids, pancreatic organoid, epithelial organoids, lung organoids, kidney organoids, gastruloids (embryonic organoids), or cardiac organoids, and/or
wherein the patient-specific cells comprise one or more cell types selected from the group consisting of cancerous cells, pre-cancerous cells, pericytes, stem cells, blood cells, immune cells, platelets, central nervous system neurons, glial cells, peripheral nervous system neurons, skeletal muscle cells, smooth muscle cells, chondrocytes, bone cells, skin cells, hepatic cells, endothelial cells, epithelial cells, cardiac cells, pancreatic cells, adipocytes, gastric cells, intestinal cells, renal cells, fibroblasts, gall bladder cells, duct cells, pneumocytes, lens cells, sensory transducer cells, autonomic neurons, gland cells, hormone secreting cells, nurse cells, germ cells, or any combination thereof.

5. (canceled)

6. (canceled)

7. The three-dimensional biomimetic platform system of claim 2, wherein the patient-specific cells are isolated from a subject suffering from a disease or a healthy subject.

8. The three-dimensional biomimetic platform system of claim 2, wherein the biocompatible substrate comprises about 0.1 wt % to about 10 wt % of collagen, and/or
wherein the collagen of the biocompatible substrate is a Type I collagen, a Type II collagen, a Type III collagen, a Type IV collagen, a Type V collagen, a Type VI collagen, a Type VII collagen, a Type VIII collagen, a Type IX collagen, a Type X collagen, a Type XI collagen, a Type XII collagen, a Type XIII collagen, a Type XIV collagen, a Type XV collagen, a Type XVI collagen, a Type XVII collagen, a Type XVIII collagen, a Type XIX collagen, a Type XX collagen, a Type XXI collagen, a Type XXII collagen, a Type XXIII collagen, a Type XXIV collagen, a Type XXV collagen, a Type XXVI collagen, a Type XXVII collagen, a Type XXVIII collagen, a Type XXIX collagen, or any mixture thereof, and/or
wherein the collagen has an elastic compressive modulus that ranges from about 3 kPa to about 40 kPa, and/or
wherein the collagen is modified with a glycosylating agent.

9. (canceled)

10. (canceled)

11. The three-dimensional biomimetic platform system of claim 8, wherein the glycosylating agent is glucose, ribose, fructose, galactose, glucose-6-Phosphate, lactose, maltose, xylose, glyceraldehyde, glutaraldehyde, cellobiose, corn syrup, maltodextrin, or dextrin.

12. (canceled)

13. The three-dimensional biomimetic platform system of claim 2, wherein the biocompatible substrate further comprises at least one non-collagen extracellular matrix component selected from the group consisting of fibronectin, laminin, hyaluronic acid, Matrix-bound nanovesicles (MBVs), elastin, proteoglycans, glycosaminoglycans (GAGs), heparan sulfate, perlecan, agrin, chondroitin sulfate, and keratan sulfate.

14. The three-dimensional biomimetic platform system of claim 2, wherein the one or more conduits have a shape selected from the group consisting of straight, curved, U-shape, zigzagged or any combination thereof, and/or wherein the one or more conduits form a vascular channel.

15. (canceled)

16. A method for producing a biomimetic platform system comprising
(a) preparing a biocompatible substrate;
(b) embedding a sacrificial material within the biocompatible substrate;
(c) degrading the sacrificial material to produce one or more conduits within the biocompatible substrate; and
(d) applying patient-specific cells to the one or more conduits within the biocompatible substrate.

17. The method of claim 16, wherein the biocompatible substrate comprises at least one collagen selected from the group consisting of Type I collagen, Type II collagen, Type III collagen, Type IV collagen, and Type V collagen, and/or one or more components selected from the group consisting of stromal vascular fraction, adipocytes, and organoids.

18. (canceled)

19. The method of claim 16, wherein the sacrificial material is selected from the group consisting of poloxamers, shellac, carbohydrate glass, polyvinyl alcohol (PVA), and gelatin microparticles, optionally wherein the poloxamers are selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407 (e.g., Pluronic® F127), poloxamer 105 Benzoate, and poloxamer 182 dibenzoate.

20. (canceled)

21. The method of claim 16, further comprising adding one or more biomolecules to the biocompatible substrate, wherein the one or more biomolecules are growth factors, blood, plasma, hormones, cytokines, enzymes, vitamins, fatty acids, and lymphokines.

22. A method for monitoring at least one biological activity of patient-specific cells ex vivo comprising
(a) culturing patient-specific cells in the biomimetic platform system of claim 2 under conditions that permit maturation of the patient-specific cells; and
(b) assaying at least one biological activity of the patient-specific cells, optionally wherein the at least one biological activity is cell viability, cell growth, cell division, apoptosis, cell migration, angiogenesis, gene expression, blood coagulation, or metastasis.

23. A method for screening the effect of a candidate agent on patient-specific cells comprising
(a) contacting the candidate agent with the biomimetic platform system of claim 2, wherein the biomimetic platform system comprises patient-specific cells that are cultured under conditions that permit maturation of the patient-specific cells; and
(b) assaying at least one biological activity of the treated patient-specific cells, optionally wherein the at least one biological activity is cell viability, cell growth, cell division, apoptosis, cell migration, angiogenesis, gene expression, blood coagulation, or metastasis.

24. The method of claim 23, wherein the treated patient-specific cells exhibit an alteration in at least one biological activity compared to that observed in untreated patient-specific cells.

25. (canceled)

26. A method for evaluating the toxicity of a candidate agent on patient-specific cells obtained from a healthy subject comprising
(a) contacting the candidate agent with the biomimetic platform system of claim 2, wherein the biomimetic platform system comprises patient-specific cells that are cultured under conditions that permit maturation of the patient-specific cells;
(b) assaying the viability of the treated patient-specific cells; and
(c) determining that the candidate agent is toxic when the treated patient-specific cells exhibit decreased viability compared to that observed in untreated patient-specific cells.

27. A method for determining the therapeutic efficacy of a candidate agent for treating a disease in a patient in need thereof comprising
(a) contacting the biomimetic platform system of claim 2 with the candidate agent, wherein the one or more conduits of the biomimetic platform system comprise patient-specific diseased cells that are cultured under conditions that permit maturation of the patient-specific diseased cells; and
(b) determining that the candidate agent is therapeutically effective when the treated patient-specific diseased cells exhibit decreased viability compared to that observed in untreated patient-specific cells.

28. The method of claim 27, wherein the disease is a cancer selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

29. The method of claim 23, wherein the candidate agent is a synthetic low-molecular-weight compound, a natural compound, a recombinant protein, a purified or crude protein, a peptide, a non-peptide compound, an antibody, an engineered cell, a vaccine, a nucleic acid (e.g., a siRNA, an antisense oligonucleotide, a sgRNA, an aptamer), a recombinant virus, a recombinant microorganism, a ribozyme, a cell extract, a cell culture supernatant, a microbial fermentation product, a marine organism extract, a plant extract, or any combination thereof.

30. The method of claim 23, wherein the candidate agent is a chemotherapeutic agent.
